SEQUENCE LISTING (54 sequences)

SEQ ID NO: 1 - DNA, 212 bp, Artificial Sequence
  Synthetic Polynucleotide
ttttttttttcggagctgagaccgaacgtaggcttcggcactgttagaagatatcaacaattcacgtatgcgcgtggtaacttgtcttttgattcactgccattctgcggagctcccattcagatccacctggaggggaaagatagtttatgtcacacagtactaacaaaaacccgggtttagtctaggcggtcctgccccgtttttttttt

SEQ ID NO: 2 - DNA, 317 bp, Artificial Sequence
  Synthetic Polynucleotide
tggccacgatccatgctaacgtctctgcgtagggatgaatcccgttttgaactcgttcctactgacggacgagctgataggtagccgaagtagtgatacgatccacacatgccatcattgcatactcgtgcattcaatgatgcatagtcacgtagtccatatggtaatggtgatgtcaagtcacatgtcaatactcgtcactagaactgagcgcgatgactggcgagctggtgcgctcccgaggctggtcgagcgactaagttgaatgcgcagaccgatcgagacgactctagcgctggaataaatcagaataaaga

SEQ ID NO: 3 - DNA, 317 bp, Artificial Sequence
  Synthetic Polynucleotide
cccaccaatactgccaatagacggtactgtacaccctgttttacagcaacgggaaaggaggatcactttctacaattgtgtgctggactgacagtcgcatatccacacatgccatcattgcatactcgtgcattcaatgatgcatctacacgtagtccatatggtaatggtgatgtcactacacatgtcaatactcgtcactagaactgagcgcgatacgactcgcccatagggttcgccggctcgcactgactaccttacgctctgacccagatcggagccggccgcatgacccctgtgatataataccgttcatc

SEQ ID NO: 4 - DNA, 525 bp, Artificial Sequence
  Synthetic Polynucleotide
taatacgactcactatagggacagtctagtgcagcagtcagtacgagtctcatgagtgtaggatgcatgatcatgattctgatctagtccagcagtagagtcgtctcgatcgatctgtgcatcgtcagcgatattcgacgtagtcgctcgacctgactcgtgagtgcagctacgtgtcagtcatccactgttgccatatatgcagacggcatagtatgcgtgtatgcgtcgagagatcatccagttcttgacgttagttacaagattggccacgatccatgctaacgtctcttccacctttcccaaaaagtaacaccgactgatcgcgcatacggcaacagtgactctcgactaccatagtagtgagatggtggattacgatcgcgtgatctgagtatcattgatctatagtggattgactgatgatcgtactgtcgtactgactctgacgtcgatctcaggtcatattactcgacagttgctaagtcagtcatcgtcatacgatgccgctgagcaataactagc

SEQ ID NO: 5 - DNA, 525 bp, Artificial Sequence
  Synthetic Polynucleotide
gctagttattgctcagcggcatcgtatgacgatgactgacttagcaactgtcgagtaatatgacctgagagctactgatctgactagctaagcttgcatgcacgtcatgatccagtatagatcaatgatactcagatcacgcgatatcgacgttgactagtcaagctagatccacatatgctgtatgtgcgtagtcgatgtcatgactatgttttacagcaacgggaaaggaggaccgtctattggcagtattggtgggatcttgtaactaacgtcaagatagggatgatctctcgacgcatacacgcattagatgccgtctgcatatatggcaacagtggatacgactcgatcatcgagttcgcatgctagcactgactacgttacgctctgatctcagacgatagtcagatcggagtcagctgcatgacgacagtgcgatgctagcgttgatctcatgcatcctacactcatgagactcgtactgactgctgcactagactgtccctatagtgagtcgtatta

SEQ ID NO: 6 - DNA, 525 bp, Artificial Sequence
  Synthetic Polynucleotide
taatacgactcactatagggacagtctagtgcagcagtcagtacgagtctcatgagtgtaggatgcatgatcatgattctgatctagtccagcagtagagtcgtctcgatcgatctgtgcatcgtcgacgatattcgacgtagtcgctcgacctgactcgtgagtgcagctacgtgtcagtcatccactgttgccatatatgcagacggcatagtatgcgtgtatgcgtcgagagatcatccagttcttgacgttagttacaagattggccacgatccatgctaacgtctcttccacctttcccaaaaagtaacacaccatgacgtatcgactacgcacatacagcatatgtggatgatcactgactgactgaactacgatcatggtgtatgtgagcgtgtatgtgctcgtgactggagaaacggcaacagtggatgattgacgtacgactgctagctcaggtcatattactcgacagttgctaagtcagtcatcgtcatacgatgccgctgagcaataactagc

SEQ ID NO: 7 - DNA, 525 bp, Artificial Sequence
  Synthetic Polynucleotide
gctagttattgctcagcggcatcgtatgacgatgactgacttagcaactgtcgagtaatatgacctgagagtcagtgctcatgatgtcaatccactgttgccgtttctccctacacgagcacatacacgctcacatacaccatgatgactagcatgatcatccaccgtgtatctagatcacgccggcatgatctgatgacgatcatgactgttttacagcaacgggaaaggaggaccgtctattggcagtattggtgggatcttgtaactaacgtcaagatagggatgatctctcgacgcatacacgcattagatgccgtctgcatatatggcaacagtggatacgactcgatcatcgagttcgcatgctagcactgactacgttacgctctgatctcggacgatagtcagatcggagtcagctgcatgacgacagtgcgatgctagcgttgatctcatgcatcctacactcatgagactcgtactgactgctgcactagactgtccctatagtgagtcgtatta

SEQ ID NO: 8 - DNA, 525 bp, Artificial Sequence
  Synthetic Polynucleotide
taatacgactcactatagggacagtctagtgcagcagtcagtacgagtctcatgagtgtaggatgcatgatcatgattctgatctagtccagcagtagagtcgtctcgatcgatctgtgcatcgtcacggatattcgacgtagtcgctcgacctgactcgtgagtgcagctacgtgtcagtcatccactgttgccatatatgcagacggcatagtatgcgtgtatgcgtcgagagatcatccagttcttgacgttagttacaagattggccacgatccatgctaacgtctcttccacctttcccaaaaagtaacactgactgcattcgtgatcatcatgccggcgtgatctagatacacggtggattcagctactagtcgaatcatgacgtgagaagcatgaacgatatgaagaagttatgtggatagctgtcgacgtgatcgtatcgatgcagtcctcaggtcatattactcgacagttgctaagtcagtcatcgtcatacgatgccgctgagcaataactagc

SEQ ID NO: 9 - DNA, 525 bp, Artificial Sequence
  Synthetic Polynucleotide
gctagttattgctcagcggcatcgtatgacgatgactgacttagcaactgtcgagtaatatgacctgagagctatcgatgacgtactgatgtcatcatgatccacataacttcttcatatcgttcatgcttctcacgtcatgataacgcatccaccatctcactactatggtagtcgagctacactgttgccgtatgcgcgatgtcaattgttttacagcaacgggaaaggaggaccgtctattggcagtattggtgggatcttgtaactaacgtcaagatagggatgatctctcgacgcatacacgcattagatgccgtctgcatatatggcaacagtggatacgactcgatcatcgagttcgcatgctagcactgactacgttacgctctgatcctagacgatagtcagatcggagtcagctgcatgacgacagtgcgatgctagcgttgatctcatgcatcctacactcatgagactcgtactgactgctgcactagactgtccctatagtgagtcgtatta

SEQ ID NO: 10 - DNA, 23 bp, Artificial Sequence
  Synthetic Polynucleotide
gacattaacctataaaaataggc

SEQ ID NO: 11 - DNA, 19 bp, Artificial Sequence
  Synthetic Polynucleotide
gcatcttccaggaaatctc

SEQ ID NO: 12 - DNA, 20 bp, Artificial Sequence
  Synthetic Polynucleotide
taatacgactcactataggg

SEQ ID NO: 13 - DNA, 19 bp, Artificial Sequence
  Synthetic Polynucleotide
gctagttattgctcagcgg

SEQ ID NO: 14 - DNA, 100 bp, Artificial Sequence
  Synthetic Polynucleotide
tggccacgatccatgctaacgtctctgcgtagggatgaatcccgttttgaactcgttcctactgacggacgagctgataggtagccgaagtagtgatacg

SEQ ID NO: 15 - DNA, 100 bp, Artificial Sequence
  Synthetic Polynucleotide
cccaccaatactgccaatagacggtactgtacaccctgttttacagcaacgggaaaggaggatcactttctacaattgtgtgctggactgacagtcgcat

SEQ ID NO: 16 - DNA, 100 bp, Artificial Sequence
  Synthetic Polynucleotide
gactggcgagctggtgcgctcccgaggctggtcgagcgactaagttgaatgcgcagaccgatcgagacgactctagcgctggaataaatcagaataaaga

SEQ ID NO: 17 - DNA, 100 bp, Artificial Sequence
  Synthetic Polynucleotide
acgactcgcccatagggttcgccggctcgcactgactaccttacgctctgacccagatcggagccggccgcatgacccctgtgatataataccgttcatc

SEQ ID NO: 18 - DNA, 525 bp, Artificial Sequence
  Synthetic Polynucleotide
taatacgactcactatagggacagtctagtgcagcagtcagtacgagtctcatgagtgtaggatgcatgagatcaacgctagcatcgcactgtcgtcatgcagctgactccgatctgactatcgtctgagatcagagcgtaacgtagtcagtgctagcatgcgaactcgatgatcgagtcgtatccactgttgccatatatgcagacggcatagtatgcgtgtatgcgtcgagagatcatccctatcttgacgttagttacaagatcccaccaatactgccaatagacggtcctcctttcccgttgctgtaaaacagtcatgatcgtcatcagatcatgccggcgtgatctagatacacggtggattcagctactagtcgaatcatgacgtgagaagcatgaacgatatgaagaagttatgtggatagctgtcgacgtgatcgtatcgatgcagtcctcaggtcatattactcgacagttgctaagtcagtcatcgtcatacgatgccgctgagcaataactagc

SEQ ID NO: 19 - DNA, 525 bp, Artificial Sequence
  Synthetic Polynucleotide
taatacgactcactatagggacagtctagtgcagcagtcagtacgagtctcatgagtgtaggatgcatgatcatgattctgatctagtccagcagtagagtcgtctcgatcgatctgtgcatcgtcagcgatattcgacgtagtcgctcgacctgactcgtgagtgcagctacgtgtcagtcatccactgttgccatatatgcagacggcatagtatgcgtgtatgcgtcgagagatcatccagttcttgacgttagttacaagattggccacgatccatgctaacgtctcttccacctttcccaaaaagtaacacaccatgacgtatcgactacgcacatacagcatatgtggatgatcactgactgactgaactacgatcatggtgtatgtgagcgtgtatgtgctcgtgactggagaaacggcaacagtggatgattgacgtacgactgctagctcaggtcatattactcgacagttgctaagtcagtcatcgtcatacgatgccgctgagcaataactagc

SEQ ID NO: 20 - DNA, 525 bp, Artificial Sequence; identical to SEQ ID NO: 4
  Synthetic Polynucleotide

SEQ ID NO: 21 - DNA, 525 bp, Artificial Sequence
  Synthetic Polynucleotide
taatacgactcactatagggacagtctagtgcagcagtcagtacgagtctcatgagtgtaggatgcatgatcatgattctgatctagtccagcagtagagtcgtctcgatcgatctgtgcatcgtcagcgatattcgacgtagtcgctcgacctgactcgtgagtgcagctacgtgtcagtcatccactgttgccatatatgcagacggcatagtatgcgtgtatgcgtcgagagatcatccagttcttgacgttagttacaagattggccacgatccatgctaacgtctcttccacctttcccaaaaagtaacactgactgcattcgtgatcatcatgccggcgtgatctagatacacggtggattcagctactagtcgaatcatgacgtgagaagcatgaacgatatgaagaagttatgtggatagctgtcgacgtgatcgtatcgatgcagtcctcaggtcatattactcgacagttgctaagtcagtcatcgtcatacgatgccgctgagcaataactagc

SEQ ID NO: 22 - DNA, 525 bp, Artificial Sequence
  Synthetic Polynucleotide
taatacgactcactatagggacagtctagtgcagcagtcagtacgagtctcatgagtgtaggatgcatgagatcaacgctagcatcgcactgtcgtcatgcagctgactccgatctgactatcgtctgagatcagagcgtaacgtagtcagtgctagcatgcgaactcgatgatcgagtcgtatccactgttgccatatatgcagacggcatagtatgcgtgtatgcgtcgagagatcatccctatcttgacgttagttacaagatcccaccaatactgccaatagacggtcctcctttcccgttgctgtaaaacatagtcatgacatcgactacgcacatacagcatatgtggatctagcttgactagtcaacgtcgatatcgcgtgatctgagtatcattgatctatagtggattgactgatgatcgtactgtcgtactgactctgacgtcgatctcaggtcatattactcgacagttgctaagtcagtcatcgtcatacgatgccgctgagcaataactagc

SEQ ID NO: 23 - DNA, 525 bp, Artificial Sequence
  Synthetic Polynucleotide
taatacgactcactatagggacagtctagtgcagcagtcagtacgagtctcatgagtgtaggatgcatgagatcaacgctagcatcgcactgtcgtcatgcagctgactccgatctgactatcgtctgagatcagagcgtaacgtagtcagtgctagcatgcgaactcgatgatcgagtcgtatccactgttgccatatatgcagacggcatagtatgcgtgtatgcgtcgagagatcatccctatcttgacgttagttacaagatcccaccaatactgccaatagacggtcctcctttcccgttgctgtaaaacatagtcatgacatcgactacgcacatacagcatatgtggatctagcttgactagtcaacgtcgatatcgcgtgatctgagtatcattgatctatagtggatcatgacgtgcatgcaagcttagctagtcagatcagtagctctcaggtcatattactcgacagttgctaagtcagtcatcgtcatacgatgccgctgagcaataactagc

SEQ ID NO: 24 - DNA, 192 bp, Artificial Sequence
  Synthetic Polynucleotide
cggagctgagaccgaacgtaggcttcggcactgttagaagatatcaacaattcacgtatgcgcgtggtaacttgtcttttgattcactgccattctgcggagctcccattcagatccacctggaggggaaagatagtttatgtcacacagtactaacaaaaacccgggtttagtctaggcggtcctgccccg

SEQ ID NO: 25 - DNA, 600 bp, Artificial Sequence
  Synthetic Polynucleotide
  misc_feature (1)..(12): n is a, c, g, or t
  misc_feature (15)..(15): n is a, c, g, or t
  misc_feature (17)..(20): n is a, c, g, or t
  misc_feature (52)..(52): n is a, c, g, or t
  misc_feature (54)..(54): n is a, c, g, or t
  misc_feature (58)..(58): n is a, c, g, or t
  misc_feature (77)..(78): n is a, c, g, or t
  misc_feature (201)..(201): n is a, c, g, or t
  misc_feature (229)..(229): n is a, c, g, or t
  misc_feature (257)..(257): n is a, c, g, or t
  misc_feature (274)..(274): n is a, c, g, or t
  misc_feature (438)..(438): n is a, c, g, or t
  misc_feature (446)..(446): n is a, c, g, or t
nnnnnnnnnnnncgncnnnnctcgagctggtggcgcgccttatttgtatagngncccnattgtgctagacggcttgnnaccctgaatcccgttttggactcgttccgctgatttctaaactggtgggcagccgagacagtgatataatcccccctggccatcttggcttactcgggctttcatggaggctctaccccgtanccctatgggaatgggggaggccctaccnctggtcataactcgtcactagaactgancgcgatgactgcggagntggggcgctccggatcgtggtggatagattacgctgagtgcccaaaccgatccagacgactctcccctgggaataaatcccaataatcacctagggatatattccgcttcctcgctcactgactcgctacgctcggccgttcgatggcggcgagcgggaatggntttacgancggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactata

SEQ ID NO: 26 - DNA, 599 bp, Artificial Sequence
  Synthetic Polynucleotide
  misc_feature (213)..(213): n is a, c, g, or t
  misc_feature (223)..(223): n is a, c, g, or t
  misc_feature (234)..(234): n is a, c, g, or t
  misc_feature (237)..(237): n is a, c, g, or t
  misc_feature (249)..(249): n is a, c, g, or t
  misc_feature (526)..(526): n is a, c, g, or t
  misc_feature (591)..(592): n is a, c, g, or t
  misc_feature (594)..(599): n is a, c, g, or t
aatctcgataactcaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagctggtggcgcgccttatttgtatagcccncccgatccancaatacgtctntgngtagggatgaanttacttcagcggtagaggaggatcacggtcgacatgataggtaggggaagtcattatacgatccacacatgccatcattgcatactcgtgcattcaatgatgcatagtcacgtagtccatatggtaatggtgatgtcaagtcacatgtcaatactcgtcactagaactgagcgcgatgcctgtcgagcatgggcgctcccgagtctcgtcgagcgcctaacgctaatgcccagatcggtgccggcgactttagccctggaataaatcagaattaagncctagggatatattccgcttcctcgctcactgactcgctacgctcggtcgttcgactgcggcganngnnnnnn

SEQ ID NO: 27 - DNA, 84 bp, Artificial Sequence
  Synthetic Polynucleotide
atccactgttgccatatatgcagacggcatagtatgcgtgtatgcgtcgagagatcatccagttcttgacgttagttacaagat

SEQ ID NO: 28 - DNA, 84 bp, Artificial Sequence
  Synthetic Polynucleotide
atccactgttgccatatatgcagacggcatctaatgcgtgtatgcgtcgagagatcatccctatcttgacgttagttacaagat

SEQ ID NO: 29 - DNA, 111 bp, Artificial Sequence
  Synthetic Polynucleotide
  misc_feature (107)..(107): n is a, c, g, or t
ctcgatgatcgagtcgcatccactgttgccatatatgcagacggcatctaatgcgtgtatgcgtcgagagatcatccctatcttgacgttagttacaagattgcacncgat

SEQ ID NO: 30 - DNA, 36 bp, Artificial Sequence
  Synthetic Polynucleotide
atccactatagatcaatgatactcagatcacgcgat

SEQ ID NO: 31 - DNA, 36 bp, Artificial Sequence; identical to SEQ ID NO: 30
  Synthetic Polynucleotide

SEQ ID NO: 32 - DNA, 72 bp, Artificial Sequence
  Synthetic Polynucleotide
aagcttgcatgcacgtcatgatccactatagatcaatgatactcagatcacgcgatatcgacgttgactagt

SEQ ID NO: 33 - DNA, 57 bp, Artificial Sequence
  Synthetic Polynucleotide
atccactgttgccgtttctccagtcacgagcacatacacgctcacatacaccatgat

SEQ ID NO: 34 - DNA, 57 bp, Artificial Sequence
  Synthetic Polynucleotide
atccactgttgccgtttctccctacacgagcacatacacgctcacatacaccatgat

SEQ ID NO: 35 - DNA, 93 bp, Artificial Sequence
  Synthetic Polynucleotide
  misc_feature (1)..(1): n is a, c, g, or t
  misc_feature (4)..(5): n is a, c, g, or t
  misc_feature (7)..(8): n is a, c, g, or t
  misc_feature (10)..(10): n is a, c, g, or t
  misc_feature (12)..(13): n is a, c, g, or t
  misc_feature (15)..(17): n is a, c, g, or t
ncanncnnangnnannnatccactgttgccgtttctccctacacgagcacatacacgctcacatacaccatgatcgcaattcagtccgtccgt

SEQ ID NO: 36 - DNA, 45 bp, Artificial Sequence
  Synthetic Polynucleotide
atccacataacttcttcatatcgttcatgcttctcacgtcatgat

SEQ ID NO: 37 - DNA, 45 bp, Artificial Sequence; identical to SEQ ID NO: 36
  Synthetic Polynucleotide

SEQ ID NO: 38 - DNA, 82 bp, Artificial Sequence
  Synthetic Polynucleotide
  misc_feature (1)..(4): n is a, c, g, or t
  misc_feature (6)..(18): n is a, c, g, or t
  misc_feature (77)..(77): n is a, c, g, or t
nnnnannnnnnnnnnnnngatccacataacttcttcatatcgttcatgcttctcacgtcatgattccgctaccaccntctcc

SEQ ID NO: 39 - DNA, 30 bp, Artificial Sequence
  Synthetic Polynucleotide
atccacatatgctgtatgtgcgtagtcgat

SEQ ID NO: 40 - DNA, 30 bp, Artificial Sequence; identical to SEQ ID NO: 39
  Synthetic Polynucleotide

SEQ ID NO: 41 - DNA, 68 bp, Artificial Sequence
  Synthetic Polynucleotide
  misc_feature (54)..(55): n is a, c, g, or t
tgactagtcagtgatcatccacatatgctgtatgtgcgtagtcgatgtcatgannagtgtttactttt

SEQ ID NO: 42 - DNA, 33 bp, Artificial Sequence
  Synthetic Polynucleotide
atccaccgtgtatctagatcacgccggcatgat

SEQ ID NO: 43 - DNA, 33 bp, Artificial Sequence; identical to SEQ ID NO: 42
  Synthetic Polynucleotide

SEQ ID NO: 44 - DNA, 84 bp, Artificial Sequence
  Synthetic Polynucleotide
tacaccatgatgactagcatgatcatccaccgtgtatctagatcacgccggcatgatctgatgacgatcatgactgttttacag

SEQ ID NO: 45 - DNA, 54 bp, Artificial Sequence
  Synthetic Polynucleotide
atccaccatctcactactatggtagtcgagagtcactgttgccgtatgcgcgat

SEQ ID NO: 46 - DNA, 54 bp, Artificial Sequence
  Synthetic Polynucleotide
atccaccatctcactactatggtagtcgagctacactgttgccgtatgcgcgat

SEQ ID NO: 47 - DNA, 89 bp, Artificial Sequence
  Synthetic Polynucleotide
acgtcatgataacgcatccaccatctcactactatggtagtcgagctacactgttgccgtatgcgcgatgtcaattgttttacagcagc

SEQ ID NO: 48 - DNA, 12 bp, Artificial Sequence
  Synthetic Polynucleotide
cagatcgatgcg

SEQ ID NO: 49 - DNA, 12 bp, Artificial Sequence
  Synthetic Polynucleotide
agtctcgagata

SEQ ID NO: 50 - DNA, 12 bp, Artificial Sequence
  Synthetic Polynucleotide
aagctcgatacg

SEQ ID NO: 51 - DNA, 117 bp, Artificial Sequence
  Synthetic Polynucleotide
atccacacatgccatcattgcatactcgtgcattcaatgatgcatagtcacgtagtccatatggtaatggtgatgtcaagtcacatgtcaatactcgtcactagaactgagcgcgat

SEQ ID NO: 52 - DNA, 117 bp, Artificial Sequence
  Synthetic Polynucleotide
atccacacatgccatcattgcatactcgtgcattcaatgatgcatctacacgtagtccatatggtaatggtgatgtcactacacatgtcaatactcgtcactagaactgagcgcgat

SEQ ID NO: 53 - DNA, 60 bp, Artificial Sequence
  Synthetic Polynucleotide
tcaatactcgtcactagaactgagcgcgatgactggcgagctggtgcgctcccgaggctg

SEQ ID NO: 54 - DNA, 60 bp, Artificial Sequence
  Synthetic Polynucleotide
tcaatactcgtcactagaactgagcgcgatacgactcgcccatagggttcgccggctcgc

(19) United States
(12) Patent Application Publication
(10) Pub. No.: US 2017/0338943 A1
(43) Pub. Date: Nov. 23, 2017

(54) DNA ENCRYPTION TECHNOLOGIES

(71) Applicant: Massachusetts Institute of Technology, Cambridge, MA (US)

(72) Inventors: Timothy Kuan-Ta Lu, Cambridge, MA (US); Peter A. Carr, Medford, MA (US); Bijan Zakeri, Revere, MA (US)

(73) Assignee: Massachusetts Institute of Technology, Cambridge, MA (US)

(21) Appl. No.: 15/521,956

(22) PCT Filed: Oct. 29, 2015
(86) PCT No.: PCT/US15/58120

§ 371 Date: Apr. 26, 2017

Related U.S. Application Data
(60) Provisional application No. 62/069,994, filed on Oct. 29, 2014.

(51) Int. Cl. H04L 9/06; G09C 1/00; C12Q 1/68

(52) U.S. Cl. CPC H04L 9/06; H04L 9/0656; C12Q 1/6869; G09C 1/00

(57) ABSTRACT

In some aspects, the instant disclosure relates to the multiplexed encryption of information on nucleic acid molecules. In some aspects, the instant disclosure relates to a method of secure communication of information disseminated across at least one nucleic acid molecule, the method comprising (a) obtaining a modified keyboard comprising a personalized platform for translating text into a nucleic acid sequence; (b) translating a quantum of information into a nucleic acid message sequence using the modified keyboard of (a); and, (c) obtaining an at least one nucleic acid molecule, each molecule comprising: (i) the complete or a portion of the nucleic acid message sequence, and (ii) at least one contiguous stretch of randomized variable nucleic acid sequence flanking and/or inserted into the message sequence, thereby producing a nucleic acid molecule or a set of nucleic acid molecules containing the entire quantum of information.

### RELATED APPLICATIONS

This application claims the benefit of U.S. provisional application Ser. No. 62/069,994, filed on Oct. 29, 2014, and entitled “DNA Encryption Technologies”, the entire content of which is incorporated herein by reference.

### FEDERALLY SPONSORED RESEARCH

This invention was made with government support under Contract No. N66001-12-C-4016 awarded by the Space and Naval Warfare Systems Center. The government has certain rights in the invention.

### BACKGROUND OF INVENTION

As the costs and time constraints of DNA synthesis and sequencing are rapidly declining, DNA is emerging as a viable medium for information storage. Previously, DNA has been used for hiding messages and storing large texts, however these methods require advanced laboratories with trained scientists to extract information. Simpler writing and reading methods are required for DNA communication to become more adopted.

### SUMMARY OF INVENTION

In some aspects, the instant disclosure relates to a method of secure communication of information disseminated across at least one nucleic acid molecule, the method comprising (a) obtaining a modified keyboard comprising a personalized platform for translating text into a nucleic acid sequence; (b) translating a quantum of information into a nucleic acid message sequence using the modified keyboard of (a); and, (c) obtaining an at least one nucleic acid molecule, each molecule comprising: (i) the complete or a portion of the nucleic acid message sequence, and (ii) at least one contiguous stretch of randomized variable nucleic acid sequence flanking and/or inserted into the message sequence, thereby producing a nucleic acid molecule or a set of nucleic acid molecules containing the entire quantum of information. In some embodiments, the nucleic acid molecules are naturally-occurring. In some embodiments, the nucleic acid molecules are synthesized or non-naturally occurring. In some embodiments, the sequences of the nucleic acids are naturally-occurring. In some embodiments, the sequences of the nucleic acid molecules are synthesized or non-naturally occurring. In some embodiments, the modified keyboard comprises codons. In some embodiments, the codons are designed to normalize frequency of character usage.

In some aspects, the instant disclosure relates to a method of secure communication of information contained on a single nucleic acid molecule, the method comprising (a) obtaining a nucleic acid molecule of known sequence; (b) obtaining a modified keyboard comprising a personalized platform for translating nucleic acid sequence into text; and, (b) generating a quantum of information translated from the nucleic acid sequence using the modified keyboard of (a). In some embodiments, the modified keyboard comprises codons. In some embodiments, the codons are designed to normalize frequency of character usage.

In some embodiments, the method further comprises co-sequencing the set of nucleic acid molecules using one or more common primers. In some embodiments, the co-sequencing produces patterns in a chromatogram. In some embodiments, the method further comprises identifying nucleic acid sequence corresponding to areas of high intensity peaks on the chromatogram. In some embodiments, the method further comprises identifying nucleic acid sequence corresponding to areas of low intensity peaks on the chromatogram. In some embodiments, co-sequencing produces no chromatogram pattern. In some embodiments, the method further comprises identifying nucleic acid sequence using sequence alignments generated by bioinformatics software. In some embodiments, the method further comprises extracting the quantum of information contained within the set of nucleic acid molecules by using the modified keyboard to translate the nucleic acid sequence from the one or more nucleic acid molecules.

In some embodiments, the modified keyboard comprises homopolymer codons. In some embodiments, the keyboard comprises homopolymer codons located on functional keys. In some embodiments, the codons are greater than 3 nucleotides in length. In some embodiments, the codons are 4, or 5, or 6, or 7, or 8, or 9, or 10, or 11, or 12, or 13, or 14, or 15, or 16, or 17, or 18 nucleotide bases in length. In some embodiments, the codons are of mixed lengths. In some embodiments, the variable nucleic acid sequence comprises contiguous homopolymer codons.

In some embodiments, the instant disclosure relates to methods of extracting a quantum of encrypted information from a plurality of nucleic acid molecules. In some embodiments, the encrypted information is extracted by nucleic acid sequencing. In some embodiments, the nucleic acid sequencing is co-sequencing. In some embodiments, the co-sequencing is DNA co-sequencing. In some embodiments, the DNA co-sequencing is performed by Sanger sequencing. In some embodiments, the plurality of nucleic acid molecules are sequenced with at least one common primer. In some embodiments, data produced from nucleic acid sequencing is analyzed by sequence alignment. In certain embodiments, the nucleic acid molecule(s) are in silico.

In some aspects, the instant disclosure relates to a method of producing an individualized keyboard for the conversion of plaintext into nucleic acid encodable language, the method comprising: (a) producing a library of codons; (b) assigning each member of the library to a different symbol; and, (c) arranging the symbols into an array, thereby producing an individualized keyboard. In some embodiments, the codons of the library are greater than three nucleotide bases in length. In some embodiments, the codons of the library are 4, or 5, or 6, or 7, or 8, or 9, or 10, or 11, or 12, or 13, or 14, or 15, or 16, or 17, or 18 nucleotide bases in length. In some embodiments, the codons of the library are of mixed lengths. In some embodiments, the symbol is selected from the group consisting of letter, number, word, punctuation mark or pictogram, logogram and/or any other relevant references to linguistic principles of different languages.

### BRIEF DESCRIPTION OF DRAWINGS

FIGS. 1A-1C depict one embodiment of the iKey platform. FIG. 1A depicts a graphical representation of one embodiment of an iKey-64, used to convert plaintext to codons for DNA transcription. Messages begin with ‘start’, finish with ‘end’, ‘forward’ and ‘reverse’ provide information on the strand containing the desired message, and ‘spacer 1’and ‘space2’ can be used to produce troughs in chromatograms. Codons can be randomized to produce one-time iKeys. FIG. 1B shows that in this embodiment, iKey-64 buttons and codons were numbered to transcribe the keyboard on to a single strand of DNA (SEQ ID NO: 24). FIG. 1C depicts this embodiment of iKey-64 transcribed on DNA (SEQ ID NO: 1). Codons were flanked by 10 Ts (SEQ ID NO: 1) to separate the start and end of the keyboard from surrounding DNA for identification.

FIGS. 2A-2E depict chromatogram patterning with Multiplexed Sequence Encryption (MuSE). FIG. 2A depicts a schematic for chromatogram patterning. When two DNA strands are co-sequenced, different overlapping nucleotides produce small peaks while identical ones produce large peaks. Peaks are kept in alignment via iKey-64. In FIG. 2A, SEQ ID NOs: 48 through 50 appear from top to bottom, respectively. FIG. 2B depicts a schematic demonstrating ‘Massachusetts Institute Technology’ being patterned with MuSE and iKey-64. FIG. 2C depicts the sequence of ‘Massachusetts Institute Technology used in FIG. 2B. In FIG. 2C, SEQ ID NOs: 51 and 52 appear from top to bottom, respectively. FIG. 2D shows DNA-1+2 are co-sequenced at equal concentrations with a common primer (arrows), chromatogram patterning is achieved during reverse (Primer_{ExternalRv}) but not forward (Primer_{ExternalFw}) sequencing due to the flanking variable DNA regions. FIG. 2E shows that chromatogram patterning can be tuned by varying the ratios of DNA-1 (light shading) and DNA-2 (dark shading).

FIGS. 3A-C show that chromatogram patterning requires the alignment of base calls to be maintained during co-sequencing of DNA strands. FIG. 3A shows a close-up of the chromatograms for forward; the consensus sequence listed below the alignment is represented by SEQ ID NO: 25. FIG. 3B shows a close-up of the chromatograms for reverse sequencing of DNA-1+2 encoding the MIT cipher shown in FIG. 2D; the consensus sequence listed below the alignment is represented by SEQ ID NO: 26. Samples were co-sequenced at equal concentrations and the arrow depicts the sequencing primer. FIG. 3C shows the sequence of upstream (SEQ ID NOs: 14-15) and downstream (SEQ ID NOs: 16-17) variable DNA regions from FIG. 2B.

FIG. 4 shows that MuSE can be tuned to discreetly encode messages in a mixed DNA population. By varying the ratios of DNA-1 (light shading) and DNA-2 (dark shading), the degree of chromatogram patterning can be tuned (FIG. 2E). When one partner is present at a lower concentration chromatogram patterning is still achieved, however the resulting chromatogram would align perfectly with the more concentrated partner. Therefore, messages may be discreetly encoded between multiple DNA strands and revealed in chromatograms, but not identified by sequence alignments. Left: alignment of chromatograms from FIG. 2E with DNA-1. Right: alignment of chromatograms from FIG. 2E with DNA-2.

FIG. 5 shows discreetly embedded messages in chromatograms. A close-up of chromatogram patterns formed with MuSE tuning (FIG. 2E). Message encoding regions (shaded box) contain single peaks while variable DNA regions (unshaded box) contain two overlapping peaks whose heights can be adjusted by varying the ratios of DNA-1 (SEQ ID NO: 2) and DNA-2 (SEQ ID NO: 3). The portions of DNA-1 and DNA-2 that are shown in the alignment are represented by SEQ ID NO: 53 and SEQ ID NO: 54.

FIGS. 6A-6B show a combinatorial cipher depicting a WWII communication. FIG. 6A shows that one embodiment of iKey-64 was used to transcribe watermarks, a key, a cipher, and a decoy message between 6 DNA strands. If the strands are sequenced according to the key (Pascal's triangle on left) with the appropriate primers, then the correct communication would be revealed. FIG. 6B shows the chromatograms of an n1×n6 matrix of strands tuned and co-sequenced with Primer_{Cipher}. Chromatogram patterning is not achieved when incorrect pairs are co-sequenced.

FIG. 7 shows combinatorial cipher readouts from the WWII communication of FIGS. 6A-6B. Tuning and co-sequencing of multiple DNA strands reveals a variety of messages depending on the primers used and the order of strands co-sequenced.

FIG. 8 shows that the combinatorial cipher of FIGS. 6A-6B does not produce chromatogram patterning if non-specific primers are used for co-sequencing. Co-sequencing of cipher and decoy message containing pairs at equal concentrations with non-specific primers that are common to all strands (Primer_{ExternalFw/Rv}) that bind outside of the information containing 525-bp region (FIG. 6A) does not produce chromatogram patterning.

FIGS. 9A-9G show an examination of the peaks produced during co-sequencing of the combinatorial WWII cipher of FIGS. 6A-6B. FIG. 9A shows DNA sequencing information (SEQ ID NOs: 27-29) and close-up chromatogram for the Key. FIGS. 9B-9D show DNA sequencing information (SEQ ID NOs: 30-38) and close-up chromatogram for the Cipher. FIGS. 9E-9G show DNA sequencing information (SEQ ID NOs: 39-47) and close-up chromatogram for the Decoy message.

FIG. 10 shows a 256 button iKey for introducing redundancies for transcribing plaintext in to a DNA encodable format. This is a theoretical design for an iKey-256 based on a four-nucleotide codon. While it is not designed to produce chromatogram patterning, iKey-256 would introduce redundancies in the transcription of plaintext on to DNA by equaling the frequencies of buttons for the letters used in English (Table 2). Increased number of ‘start’, ‘end’, ‘shift’, and ‘space’ buttons were implemented to reduce the overuse of any individual codon. To highlight the start and end of any message from the surrounding DNA, all 5 ‘start’ and ‘end’ codons may be used together to identify messages written within even a genome. Furthermore, a ‘I’ button was introduced to replace all punctuation characters as offline communication by DNA need not abide by grammatical rules.

FIGS. 11A-11B show DNA-based communication. FIG. 11A provides an example of NDA communication in which for Alice to send a message (m) to Bob, she must first write the data into DNA and then physically send the DNA to Bob, who can read the DNA and extract the data. Eve, who is eavesdropping, can physically intercept and read m, making the communication channel unsecure. Three areas that can improve communication between Alice and Bob include data encoding, data transfer, and data extraction. FIG. 11B provides an example of improved DNA communication. Data encoding: m can be mixed with decoy (d) data and fragmented, then written into DNA with one-time pad encryption, where the key (k) can itself be written in DNA. Data transfer: DNA encoded k and fragmented m+d components can be transmitted between Alice and Bob using multiple different channels based on a secret-sharing system. Interception of an incomplete set of DNA communications by Eve will not provide the data in m. Data extraction: chromatogram patterning can be used by Bob to rapidly extract data via multiplexed sequencing reactions.

FIGS. 12A-12C show naive co-sequencing of multiple DNA strands. FIG. 12A shows DNA-1 (top), n1(second from top), and iKey-64 (third from top) strands have different sequences but they all share a common upstream region and sequencing primer (Primer_{ExternalFw}). Individual sequencing of each strand produces high quality reads, but the resulting reads are of poor quality when two (e.g., DNA-1 and n1) or three (e.g., DNA-1, n1, and iKey64) strands are co-sequenced. FIG. 11B depicts a close-up of the chromatogram of DNA-1 (SEQ ID NO: 2) and n1 (SEQ ID NO: 4) co-sequencing. FIG. 11C depicts a close-up of the chromatogram of DNA-1, n1, and iKey64 co-sequencing (SEQ ID NOs: 2, 4 and 1, respectively).

FIG. 13 shows an example of a workflow of extracting the correct message from a DNA communication that incorporates the iKey, MuSE, and chromatogram patterning techniques. Workflow steps 1, 2, and 3 can be viewed in detail in FIGS. 6A-6B and FIG. 14. Data containing strands are pooled and sequenced with Primer_{Key} to reveal the combination key. Deciphering and unlocking of the combination key will reveal the correct strand pairs to analyze with Primer_{Message} to reveal the message. Analysis of incorrect strand pairs will reveal a decoy communication.

FIG. 14 shows an example of a combinatorial message depicting a WWII communication. iKey-64 (Encryption Key) was used to write watermarks, a key, a message, and a decoy between six DNA strands (Secret-Sharing System). If strands are sequenced according to the Combination Key—obtained from Pascal's triangle—with the appropriate primers, then the correct communication is revealed.

FIG. 15 shows an example of DNA camouflage. The 525 bp information-encoding regions of DNA were flipped between the forward and reverse strands to provide a camouflage effect against sequencing with random primer (Primer_{ExternalFw/Rv}). While the external DNA regions surrounding the information containing regions were identical, strands n1/n3/n5 were encoded in the forward direction and strands n2/n4/n6 in the reverse direction, with watermarks used for orientation.

FIGS. 16A-16C show an example of next-generation sequencing of a communication disseminated across six DNA strands. FIG. 16A shows plasmids containing n1, n2, n3, n4, n5, and n6 sequences (FIG. 15) were grown and purified in dH₂O, mixed at equal concentrations of 30 ng/μL, and submitted to an outside party for NGS sequencing and assembly under blind experimental conditions. FIG. 15B shows 300 ng of plasmids containing n1, n2, n3, n4,n5, and n6 sequences run on a 1% agarose gel to demonstrate purity. FIG. 16C shows the outside party was provided with the number of plasmids, vector sequences, and the size of messages inserted into the vectors and asked to assemble the messages encoded in the plasmids. They assembled 6 sequences (Table 5) that represent the messages n1, n2, n3, n4, n5, and n6. Here the alignment of the 6 assembled sequences with n1, n2, n3, n4, n5, and n6 are shown. Shown below the alignment is a legend for the color-coding of the templates. Boxes highlight assembled sequences with near perfect alignment to corresponding templates.

### DETAILED DESCRIPTION OF INVENTION

In some embodiments, methods are provided herein for the storage, transfer and retrieval of encrypted information within at least one nucleic acid molecule In some aspects, the instant disclosure relates to a method of secure communication of information disseminated across at least one nucleic acid molecule, the method comprising (a) obtaining a modified keyboard comprising a personalized platform for translating text into a nucleic acid sequence; (b) translating a quantum of information into a nucleic acid message sequence using the modified keyboard of (a); and, (c) obtaining at least one nucleic acid molecule, each molecule comprising: (i) the complete or a portion of the nucleic acid message sequence, and (ii) at least one contiguous stretch of randomized variable nucleic acid sequence flanking and/or inserted into the message sequence, thereby producing a nucleic acid molecule or a set of nucleic acid molecules containing the entire quantum of information. In some embodiments, the nucleic acid molecules are naturally-occurring. In some embodiments, the nucleic acid molecules are synthesized or non-naturally occurring. In some embodiments, the sequences of the nucleic acids are naturally-occurring. In some embodiments, the sequences of the nucleic acid molecules are synthesized or non-naturally occurring.

In some aspects, the instant disclosure relates to a method of secure communication of information contained on a single nucleic acid molecule, the method comprising (a) obtaining a nucleic acid molecule of known sequence; (b) obtaining a modified keyboard comprising a personalized platform for translating nucleic acid sequence into text; and, (b) generating a quantum of information translated from the nucleic acid sequence using the modified keyboard of (a).

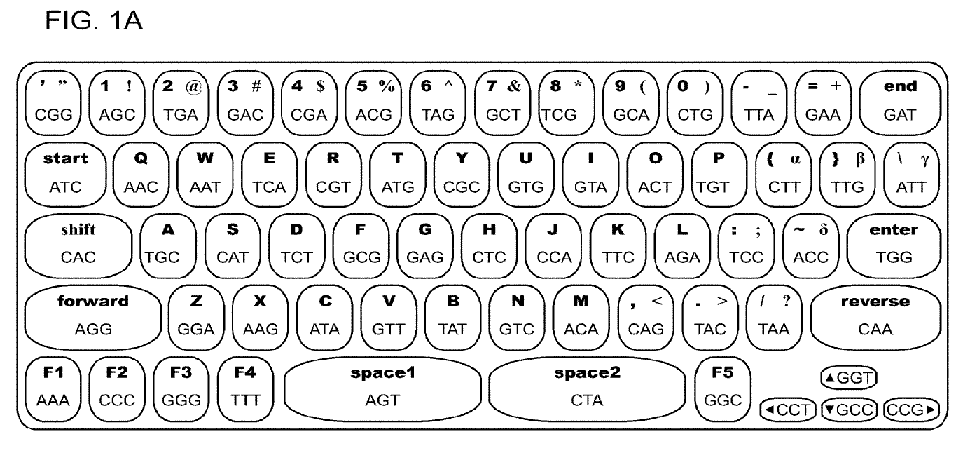

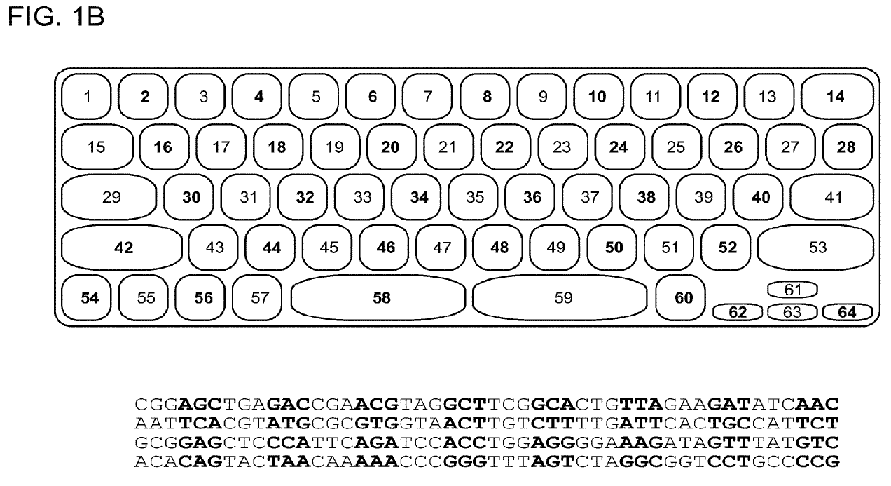

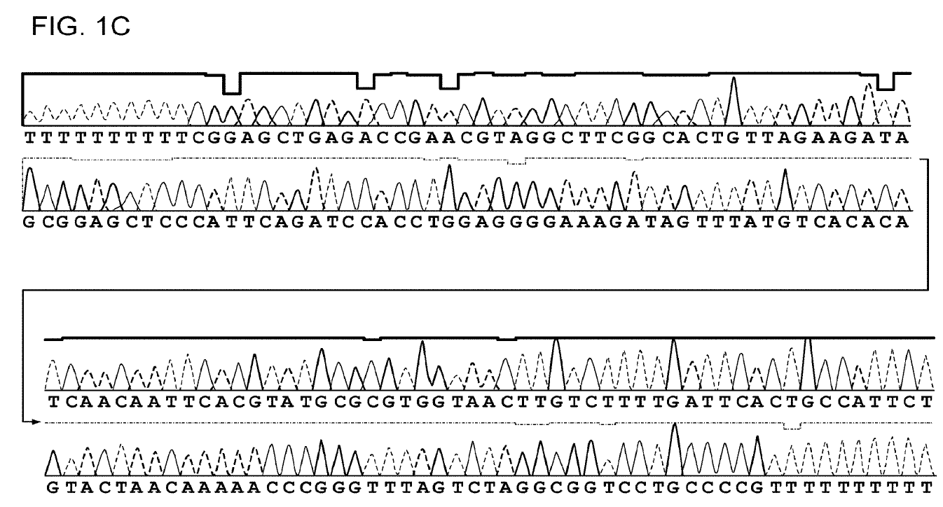

In certain aspects, the instant disclosure relates to the use of a keyboard to encrypt text information into nucleic acid sequence. For example, the keyboard can be a modified keyboard, in which the keys are modified relative to a standard “QWERTY” keyboard such that each key corresponds to specific combination of nucleotides. In some embodiments, the modified keyboard is used as a “one-time pad”. As used herein, a “one-time pad” refers to a device for the encryption of information, wherein each character of a plaintext (e.g., information) is encrypted by combining it with the corresponding bit or character of a single-use, random, secret pad or key (e.g., a modified keyboard) using modular addition. In some embodiments, the keyboard disclosed herein is a physical keyboard comprising a set of keys, wherein each key is associated with a particular codon. In some embodiments, the modified keyboard comprises homopolymer codons. In some embodiments, the keyboard comprises homopolymer codons located on functional keys. In some embodiments, homopolymer codons are associated only with functional keys. As used herein, a “functional key” refers to a key that does not translate a letter, number, word, punctuation mark or pictogram, logogram and/or any other relevant references to linguistic principles of different languages. In some embodiments, the keyboard is a virtual keyboard comprising a set of keys, wherein each key is associated with a particular codon. As used herein, a “virtual keyboard” is a keyboard appearing on a computer screen, the keys of which may be activated by a user clicking a mouse or contacting a touch screen. In some aspects, the instant disclosure relates to a method of producing an individualized keyboard for the conversion of plaintext into nucleic acid encodable language, the method comprising: (a) producing a library of codons; (b) assigning each member of the library to a different symbol; and, (c) arranging the symbols into an array, thereby producing an individualized keyboard. In some embodiments, the codons of the library are three nucleotide bases in length, such as those depicted in FIG. 1A. In some embodiments, the codons of the library are greater than three nucleotide bases in length. In some embodiments, the codons of the library are 4, or 5, or 6, or 7, or 8, or 9, or 10, or 11, or 12, or 13, or 14, or 15, or 16, or 17, or 18 nucleotide bases in length. In some embodiments, the codons of the library are of mixed lengths. In some embodiments, the symbol is selected from the group consisting of letter, number, word, punctuation mark or pictogram, logogram and/or any other relevant references to linguistic principles of different languages.

As used herein “nucleic acid” refers to a DNA or RNA molecule. Nucleic acids are polymeric macromolecules comprising a plurality of nucleotides. In some embodiments, the nucleotides are deoxyribonucleotides or ribonucleotides. In some embodiments, the nucleotides comprising the nucleic acid are selected from the group consisting of adenine, guanine, cytosine, thymine, uracil and inosine. In some embodiments, the nucleotides comprising the nucleic acid are modified nucleotides. Methods of modifying nucleotides are generally known in the art. Non-limiting examples of nucleotide modifications include phosphorothioate backbone modifications, 2′-O-methyl group sugar modifications and the substitution of non-naturally occurring nucleotide bases (for example, nucleotides derivatized at the 5-, 6-, 7- or 8-position). In some embodiments, the nucleotide modification is fusion of DNA terminal ends with at least one protein. In some embodiments, the nucleic acids of the instant disclosure are natural. Non-limiting examples of natural nucleic acids include genomic DNA, and plasmid DNA. In some embodiments, the nucleic acids of the instant disclosure are synthetic. As used herein, the term “synthetic nucleic acid” refers to a nucleic acid molecule that is constructed via the joining nucleotides by a synthetic or non-natural method. One non-limiting example of a synthetic method is solid-phase oligonucleotide synthesis. In some embodiments, the nucleic acids of the instant disclosure are isolated.

Aspects of the instant disclosure relate to the translation of information into nucleic acid sequence. In some embodiments, the amount of information to be translated into nucleic acid sequence may be measured as a quantum. As used herein, a “quantum of information” refers to a pre-determined amount of information that is expressed in the appropriate unit. Non-limiting examples of appropriate units include characters, letters, words, phrases, sentences, numbers and symbols. In some embodiments, nucleic acid sequence that comprises translated information is referred to herein as “nucleic acid message sequence”. In some embodiments, information may be translated into nucleic acid sequence using codons. As used herein, “codon” refers to a group of consecutive nucleotides that form a single unit of genetic code. Naturally-occurring codons are three nucleotides in length and represent the 20 common amino acids used to build proteins. In some embodiments, the codons used to translate information into DNA sequence are naturally-occurring codons that comprise three nucleotides. In some embodiments, the codons used to translate information into DNA sequence are greater than 3 nucleotides in length. In some embodiments, the codons are 4, or 5, or 6, or 7, or 8, or 9, or 10, or 11, or 12, or 13, or 14, or 15, or 16, or 17, or 18 nucleotide bases in length. In some embodiments, the codons are of mixed lengths. Also contemplated herein is the use of homopolymer codons. The term “homopolymer” describes a codon consisting essentially of a homogenous population of nucleotides. In some embodiments, homopolymer codons may be represented by the formulae including but not limited to [A]ₙ, [C]ₙ, [G]ₙ, [T]ₙ, [U]ₙ and [I]ₙ, wherein n is an integer representing the length of the codon. Further non-limiting examples of homopolymer codons include AAA, GGG, CCC, TTT, GGG, UUU, III, AAAA, GGGG, TTTT, CCCC, UUUU, and IIII. In some embodiments, the modified keyboards disclosed herein comprises homopolymer codons. In some embodiments, the homopolymer codons are located on the functional keys of a modified keyboard.

In some aspects, the instant disclosure relates to methods of secure communication of information by translation of said information into nucleic acid sequence. In some embodiments, the nucleic acid sequence is natural or naturally-occurring. In some embodiments, the nucleic acid sequence is synthetic or synthesized. In order to further obscure the identity of translated information, the translated information may be camouflaged within larger fragments of natural genomic or plasmid nucleic acid sequence, or variable nucleic acid sequence, to produce an encrypted nucleic acid molecule. In some embodiments, the synthesized nucleic acid molecules comprise nucleic acid message sequence and at least one contiguous stretch of randomized variable nucleic acid sequence. In some embodiments, the synthesized nucleic acid molecules comprise nucleic acid message sequence and no randomized variable nucleic acid sequence. As used herein “variable” refers to randomized nucleic acid sequence that does not comprise nucleic acid message sequence. In some embodiments, variable DNA sequence camouflages information translated into nucleic acid sequence by disrupting the fidelity of base calling during nucleic acid sequencing. In some embodiments, the variable nucleic acid sequence of the instant disclosure comprises one or more homopolymer codons. In some aspects, the presence of homopolymer codons in variable nucleic acid sequence causes an intentional misalignment of nucleic acid sequences during sequence analysis. Such misalignment may be useful in disguising the location of the encrypted information.

In some embodiments, the instant disclosure relates to methods of extracting a quantum of encrypted information from a one or more of nucleic acid molecules. In some embodiments, the encrypted information is extracted by nucleic acid sequencing. In some embodiments, the nucleic acid sequencing is co-sequencing. In some embodiments, the co-sequencing is DNA co-sequencing. In some embodiments, the DNA co-sequencing is performed by Sanger sequencing. Other non-limiting methods of DNA co-sequencing include Maxam-Gilbert sequencing, bridge PCR, nanopore sequencing and Next Generation Sequencing (e.g., Single-molecule real-time sequencing, Ion Torrent sequencing, pyrosequencing, Illumina sequencing, sequencing by ligation (SOLiD)). In some embodiments, the plurality of nucleic acid molecules are sequenced with at least one common primer. In some embodiments, the plurality of nucleic acid molecules are sequenced with 2, or 3, or 4, or 5, or 6, or 7, or 8, or 9, or 10 common primers.

In some embodiments, the method further comprises co-sequencing the set of nucleic acid molecules using one or more common primers to produce a chromatogram. A “chromatogram” refers to a visual representation of a DNA sample produced by a sequencing machine. Chromatograms depict a sequence of nucleic acid base calls as a series of peaks along a histogram. In some embodiments, the method described herein further comprises identifying information translated into nucleic acid sequence corresponding to areas of high intensity peaks on the chromatogram. In some embodiments, the method further comprises identifying nucleic acid sequence corresponding to areas of low intensity peaks on the chromatogram. In some embodiments, nucleic acid sequencing produces no chromatogram pattern. In some embodiments, the method further comprises identifying nucleic acid sequence using sequence alignments generated by bioinformatics software. In some embodiments, the method further comprises extracting the information contained within a single nucleic acid molecule or the set of nucleic acid molecules by using the modified keyboard to translate the nucleic acid sequence from the at least one nucleic acid molecule.

In some embodiments, the nucleic acid sequences and molecules described herein are in silico. As used herein, the term “in silico” refers to nucleic acid sequences or molecules produced by means of computer modeling or computer simulation. Without being bound by any particular theory, the instant disclosure contemplates the utility of in silico nucleic acid sequences and molecules for the nucleic acid encryption methods described herein. In some embodiments, in silico nucleic acid molecules or sequences may be encrypted using the methods described herein. In some embodiments, encrypted in silico nucleic acid molecules or sequences are useful for the archiving and protection of digital data.

### EXAMPLES

### Example 1

### Materials and Methods

### Plasmids

Constructs were cloned using standard molecular biology techniques, where KOD Hot Start DNA Polymerase (VWR) was used for all PCRs with primers from IDT. Synthetic DNA sequences were purchased as gBlocks from IDT (Table 1) and assembled with PCR amplified p15A origin and chloramphenicol resistance gene fusions using Gibson assembly with 25 bp sequence overlaps, either with a commercial kit (NEB) or homemade mixture²⁴, and transformed in to E. coli DH5αPRO (F⁻ φ80lacZΔM15 Δ(lacZYA-argF)U169 deoR recA1 endA1 hsdR17(rk⁻, mk⁺) phoA supE44 thi-1 gyrA96 relA1 λ⁻, PN25/tet^{R}, Placiq/lacI, Sp^{r}). Random DNA sequences were generated at http://www.bioinformatics.org/sms2/random_dna.html. All constructs were sequence verified by Genewiz Inc. (Cambridge, Mass.).

### Sequencing

All constructs (Table 1) were purified using Qiagen kits and stored in cell culture grade water (Cellgro). Constructs were diluted to a final concentration of 30 ng/μL and sent for sequencing at indicated concentrations. Primer_{ExternalFw} (GACATTAACCTATAAAAATAGGC) (SEQ ID NO: 10), Primer_{ExternalRv} (GCATCTTCCAGGAAATCTC) (SEQ ID NO: 11), Primer_{Key} (TAATACGACTCACTATAGGG) (SEQ ID NO: 12), and Primer_{Cipher} (GCTAGTTATTGCTCAGCGG) (SEQ ID NO: 13) were used for all sequencing reactions as indicated. Sequencing reactions were all performed by Genewiz Inc. (Cambridge, Mass.) under ‘Difficult Template’ settings to ensure stringent sequencing conditions were employed. All sequencing reactions were performed in triplicate. Genewiz Inc. was not consulted prior, during, or after this study and all Sager sequencing reactions were performed under blind conditions by Genewiz Inc. to ensure bias was not introduced in the results. Geneious Pro 5.5.8 was used to analyze chromatograms, perform ClustalW alignments, and produce figures.

TABLE 1
DNA Constructs

| | | | Seq |
|---|---|---|---|
| Construct | Plasmid | Sequence | ID NO: |
| iKey-64 | pBZ38 | TTTTTTTTTTCGGAGCTGAGACCGAACGTAGGCTTCGGCACTGTTAGAAGATATCAACAATTCACGTATGC | 1 |
| | | GCGTGGTAACTTGTCTTTTGATTCACTGCCATTCTGCGGAGCTCCCATTCAGATCCACCTGGAGGGGAAAG | |
| | | ATAGTTTATGTCACACAGTACTAACAAAAACCCGGGTTTAGTCTAGGCGGTCCTGCCCCGTTTTTTTTTT [symbol] | |
| DNA1 | pBZ27 | TGGCCACGATCCATGCTAACGTCTCTGCGTAGGGATGAATCCCGTTTTGAACTCGTTCCTACTGACGGACG | 2 |
| | | AGCTGATAGGTAGCCGAAGTAGTGATACGATCCACACATGCCATCATTGCATACTCGTGCATTCAATGATG | |
| | | CATAGTCACGTAGTCCATATGGTAATGGTGATGTCAAGTCACATGTCAATACTCGTCACTAGAACTGAGCG | |
| | | CGATGACTGGCGAGCTGGTGCGCTCCCGAGGCTGGTCGAGCGACTAAGTTGAATGCGCAGACCGATCGAGA | |
| | | CGACTCTAGCGCTGGAATAAATCAGAATAAAGA [symbol] | |
| DNA2 | pBZ28 | CCCACCAATACTGCCAATAGACGGTACTGTACACCCTGTTTTACAGCAACGGGAAAGGAGGATCACTTTCT | 3 |
| | | ACAATTGTGTGCTGGACTGACAGTCGCATATCCACACATGCCATCATTGCATACTCGTGCATTCAATGATG | |
| | | CATCTACACGTAGTCCATATGGTAATGGTGATGTCACTACACATGTCAATACTCGTCACTAGAACTGAGCG | |
| | | CGATACGACTCGCCCATAGGGTTCGCCGGCTCGCACTGACTACCTTACGCTCTGACCCAGATCGGAGCCGG | |
| | | CCGCATGACCCCTGTGATATAATACCGTTCATC [symbol] | |
| n1 | pBZ29 | TAATACGACTCACTATAGGGACAGTCTAGTGCAGCAGTCAGTACGAGTCTCATGAGTGTAGGATGCATGAT | 4 |
| | | CATGATTCTGATCTAGTCCAGCAGTAGAGTCGTCTCGATCGATCTGTGCATCGTCAGCGATATTCGACGTA | |
| | | GTCGCTCGACCTGACTCGTGAGTGCAGCTACGTGTCAGTCATCCACTGTTGCCATATATGCAGACGGCATA | |
| | | GTATGCGTGTATGCGTCGAGAGATCATCCAGTTCTTGACGTTAGTTACAAGATTGGCCACGATCCATGCTA | |
| | | ACGTCTCTTCCACCTTTCCCAAAAAGTAACACCGACTGATCGCGCATACGGCAACAGTGACTCTCGACTAC | |
| | | CATAGTAGTGAGATGGTGGATTACGATCGCGTGATCTGAGTATCATTGATCTATAGTGGATTGACTGATGA | |
| | | TCGTACTGTCGTACTGACTCTGACGTCGATCTCAGGTCATATTACTCGACAGTTGCTAAGTCAGTCATCGT | |
| | | CATACGATGCCGCTGAGCAATAACTAGC [symbol] | |
| n2 | pBZ30 | GCTAGTTATTGCTCAGCGGCATCGTATGACGATGACTGACTTAGCAACTGTCGAGTAATATGACCTGAGAG | 5 |
| | | CTACTGATCTGACTAGCTAAGCTTGCATGCACGTCATGATCCACTATAGATCAATGATACTCAGATCACGC | |
| | | GATATCGACGTTGACTAGTCAAGCTAGATCCACATATGCTGTATGTGCGTAGTCGATGTCATGACTATGTT | |
| | | TTACAGCAACGGGAAAGGAGGACCGTCTATTGGCAGTATTGGTGGGATCTTGTAACTAACGTCAAGATAGG | |
| | | GATGATCTCTCGACGCATACACGCATTAGATGCCGTCTGCATATATGGCAACAGTGGATACGACTCGATCA | |
| | | TCGAGTTCGCATGCTAGCACTGACTACGTTACGCTCTGATCTCAGACGATAGTCAGATCGGAGTCAGCTGC | |
| | | ATGACGACAGTGCGATGCTAGCGTTGATCTCATGCATCCTACACTCATGAGACTCGTACTGACTGCTGCAC | |
| | | TAGACTGTCCCTATAGTGAGTCGTATTA [symbol] | |
| n3 | pBZ31 | TAATACGACTCACTATAGGGACAGTCTAGTGCAGCAGTCAGTACGAGTCTCATGAGTGTAGGATGCATGAT | 6 |
| | | CATGATTCTGATCTAGTCCAGCAGTAGAGTCGTCTCGATCGATCTGTGCATCGTCGACGATATTCGACGTA | |
| | | GTCGCTCGACCTGACTCGTGAGTGCAGCTACGTGTCAGTCATCCACTGTTGCCATATATGCAGACGGCATA | |
| | | GTATGCGTGTATGCGTCGAGAGATCATCCAGTTCTTGACGTTAGTTACAAGATTGGCCACGATCCATGCTA | |
| | | ACGTCTCTTCCACCTTTCCCAAAAAGTAACACACCATGACGTATCGACTACGCACATACAGCATATGTGGA | |
| | | TGATCACTGACTGACTGAACTACGATCATGGTGTATGTGAGCGTGTATGTGCTCGTGACTGGAGAAACGGC | |
| | | AACAGTGGATGATTGACGTACGACTGCTAGCTCAGGTCATATTACTCGACAGTTGCTAAGTCAGTCATCGT | |
| | | CATACGATGCCGCTGAGCAATAACTAGC [symbol] | |
| n4 | pBZ32 | GCTAGTTATTGCTCAGCGGCATCGTATGACGATGACTGACTTAGCAACTGTCGAGTAATATGACCTGAGAG | 7 |
| | | TCAGTGCTCATGATGTCAATCCACTGTTGCCGTTTCTCCCTACACGAGCACATACACGCTCACATACACCA | |
| | | TGATGACTAGCATGATCATCCACCGTGTATCTAGATCACGCCGGCATGATCTGATGACGATCATGACTGTT | |
| | | TTACAGCAACGGGAAAGGAGGACCGTCTATTGGCAGTATTGGTGGGATCTTGTAACTAACGTCAAGATAGG | |
| | | GATGATCTCTCGACGCATACACGCATTAGATGCCGTCTGCATATATGGCAACAGTGGATACGACTCGATCA | |
| | | TCGAGTTCGCATGCTAGCACTGACTACGTTACGCTCTGATCTCGGACGATAGTCAGATCGGAGTCAGCTGC | |
| | | ATGACGACAGTGCGATGCTAGCGTTGATCTCATGCATCCTACACTCATGAGACTCGTACTGACTGCTGCAC | |
| | | TAGACTGTCCCTATAGTGAGTCGTATTA [symbol] | |
| n5 | pBZ33 | TAATACGACTCACTATAGGGACAGTCTAGTGCAGCAGTCAGTACGAGTCTCATGAGTGTAGGATGCATGAT | 8 |
| | | CATGATTCTGATCTAGTCCAGCAGTAGAGTCGTCTCGATCGATCTGTGCATCGTCACGGATATTCGACGTA | |
| | | GTCGCTCGACCTGACTCGTGAGTGCAGCTACGTGTCAGTCATCCACTGTTGCCATATATGCAGACGGCATA | |
| | | GTATGCGTGTATGCGTCGAGAGATCATCCAGTTCTTGACGTTAGTTACAAGATTGGCCACGATCCATGCTA | |
| | | ACGTCTCTTCCACCTTTCCCAAAAAGTAACACTGACTGCATTCGTGATCATCATGCCGGCGTGATCTAGAT | |
| | | ACACGGTGGATTCAGCTACTACTCCAATCATGACCTGAGAACCATGAACCATATGAAGAAGTTATGTGGAT | |
| | | AGCTGTCGACGTGATCGTATCGATGCAGTCCTCAGGTCATATTACTCGACAGTTGCTAAGTCAGTCATCGT | |
| | | CATACGATGCCGCTGAGCAATAACTAGC [symbol] | |
| n4 | pBZ37 | GCTAGTTATTGCTCAGCGGCATCGTATGACGATGACTGACTTAGCAACTGTCGAGTAATATGACCTGAGAG | 9 |
| | | CTATCGATGACGTACTGATGTCATCATGATCCACATAACTTCTTCATATCGTTCATGCTTCTCACGTCATG | |
| | | ATAACGCATCCACCATCTCACTACTATGGTAGTCGAGCTACACTGTTGCCGTATGCGCGATGTCAATTGTT | |
| | | TTACAGCAACGGGAAAGGAGGACCGTCTATTGGCAGTATTGGTGGGATCTTGTAACTAACGTCAAGATAGG | |
| | | GATGATCTCTCGACGCATACACGCATTAGATGCCGTCTGCATATATGGCAACAGTGGATACGACTCGATCA | |
| | | TCGAGTTCGCATGCTAGCACTGACTACGTTACGCTCTGATCCTAGACGATAGTCAGATCGGAGTCAGCTGC | |
| | | ATGACGACAGTGCGATGCTAGCGTTGATCTCATGCATCCTACACTCATGAGACTCGTACTGACTGCTGCAC | |
| | | TAGACTGTCCCTATAGTGAGTCGTATTA [symbol] | |

[symbol]

indicates data missing or illegible when filed

### Example 2

### Secure Offline Communication Via DNA Linguistics

### Introduction

The Internet has revolutionized communication with its great speed and volume but remains vulnerable to security breaches. For certain applications where security supersedes speed, the offline transfer of data remains vital. Moving beyond pen and paper, DNA is increasing being used as a medium for information storage and communication¹⁻⁶, and DNA cryptography and steganography have emerged as platforms for securing embedded information against unauthorized individuals⁷⁻¹⁰.

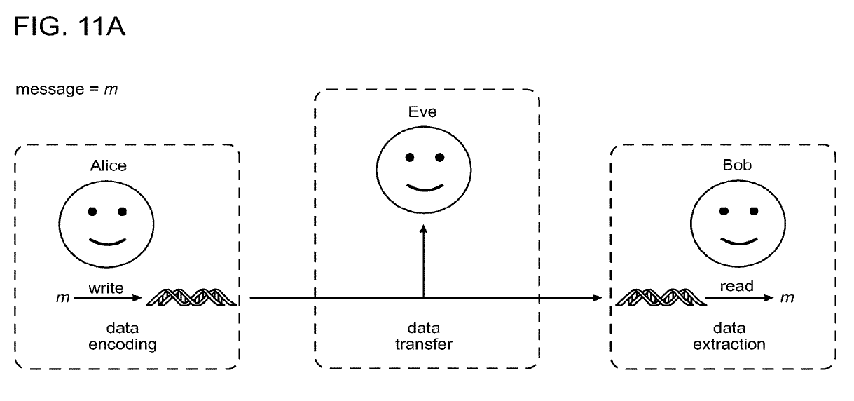

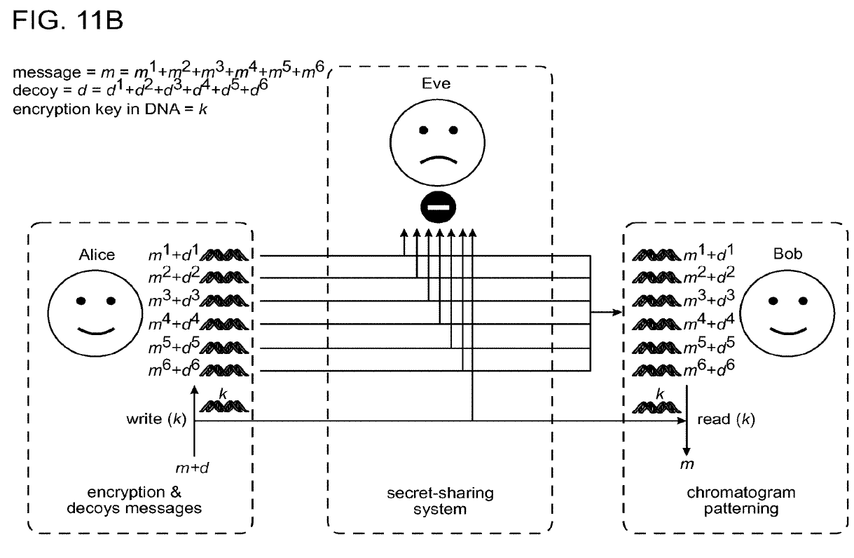

Three important points of a communication have been investigated—data encoding, data transfer & data extraction—to develop new innovations specifically for DNA-based communications (FIG. 11A). To illustrate, if Alice sends a message (m) to Bob, she would first write—encode and synthesize—the information in DNA molecules and send it to Bob who would then read—sequence and decode—the message (m). However, during the transfer of m between Alice and Bob, Eve could intercept the communication and read m. To protect m, DNA-specific cryptography and steganography methods may be implemented, however many of these methods are experimentally unproven and do not make accommodations for challenges in DNA synthesis and sequencing, such as minimizing homopolymeric stretches.

Here a new framework for the facile and secure communication of short messages in DNA is presented (FIG. 11B). To securely encode data, an encryption key (k)—that functions as a one-time pad—and decoys (d), where k is required to decode the message (m) and a combination key is required to discern m from d was implemented. To securely transfer data, a secret-sharing system was established, where m can be dispersed throughout a mixture of different DNA molecules, requiring Eve to physically intercept and interrogate multiple separate data transmission lines to gain access to m. To facilitate data extraction, chromatogram patterning, a method that allows the bypassing of sequence alignments and instead permits information to be extracted from multiple DNA molecules in a single sequencing reaction was developed.

Taking inspiration from one-time pads, considered to be an unbreakable form of encryption¹¹⁻¹⁵, described herein is a rationally designed individualized keyboard (iKey) that is amenable to randomization, serves as a facile platform to transfer plaintext on to DNA, and can achieve chromatogram patterning through co-sequencing of multiple DNA strands. Using an iKey, the secret-sharing Multiplexed Sequence Encryption (MuSE) system was developed for the secure offline communication of information that is disseminated across multiple DNA strands but can be extracted in one step. By recreating a World War II communication from Bletchley Park, it is demonstrated herein that watermarks, a key, a cipher, and a decoy can be written on DNA and the correct information is revealed only if specific strands are co-sequenced.

Development of iKey and MuSE

Here, the familiarity of text-based communication, the QWERTY keyboard, and the genetic code were combined to develop an iKey that serves as a facile platform for DNA communication.

The natural genetic code employs three-letter DNA words (codons) to represent the 20 common amino acids used to build proteins. The four-letter DNA alphabet of adenine (A), cytosine (C), guanine (G) and thymine (T) thus yields 4³=64 codons. These 64 codons were mapped onto a modified QWERTY keyboard to produce a personalized platform—iKey-64—for translating text on to DNA (FIG. 1A). The codons in iKey-64 can be randomized to produce a unique iKey for every message to provide additional security for communications, akin to a one-time pad¹¹. Any specific version of iKey-64 can itself be encoded in DNA and provided as an additional component of a communication, where it can serve as a unique dictionary for each message (FIGS. 1B-1C).

To increase the security of encoded messages in addition to the substitution cipher of iKey-64, texts were disseminated between multiple DNA strands so that the desired message would be revealed only if the correct strand combinations were analyzed. This multiplexing is at the heart of the MuSE strategy, which is a secret-sharing system where a message can be stored securely by being fragmented and distributed between multiple parties¹⁶. Analyzing only a single strand would yield either nonsense or incorrect messages designed to mislead unauthorized individuals.

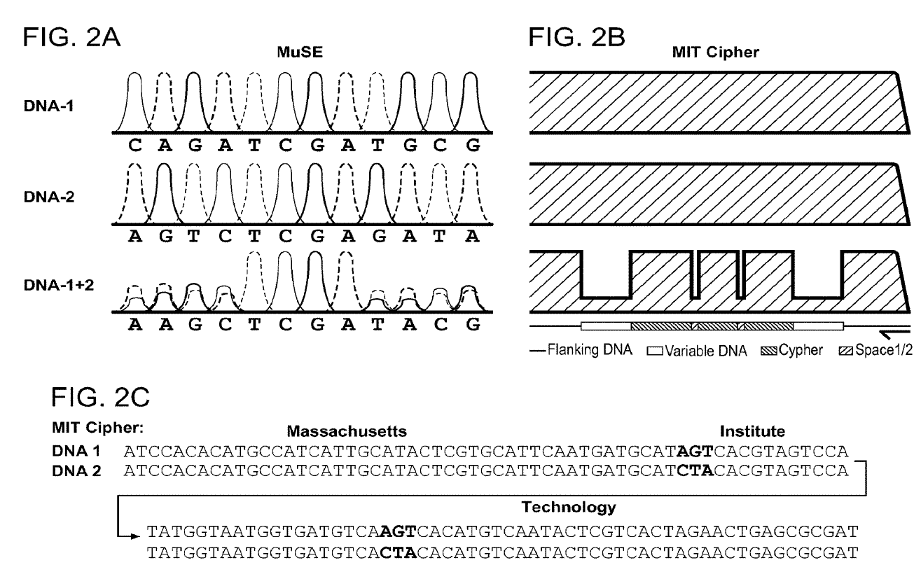

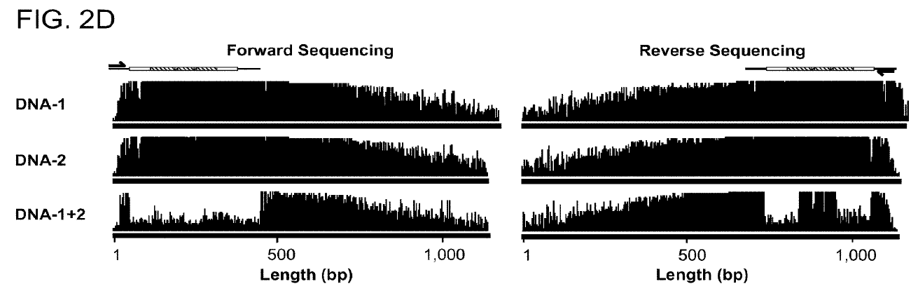

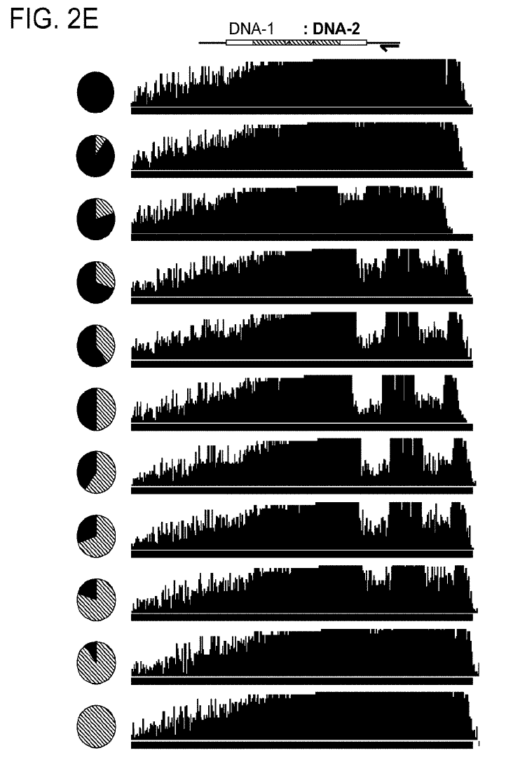

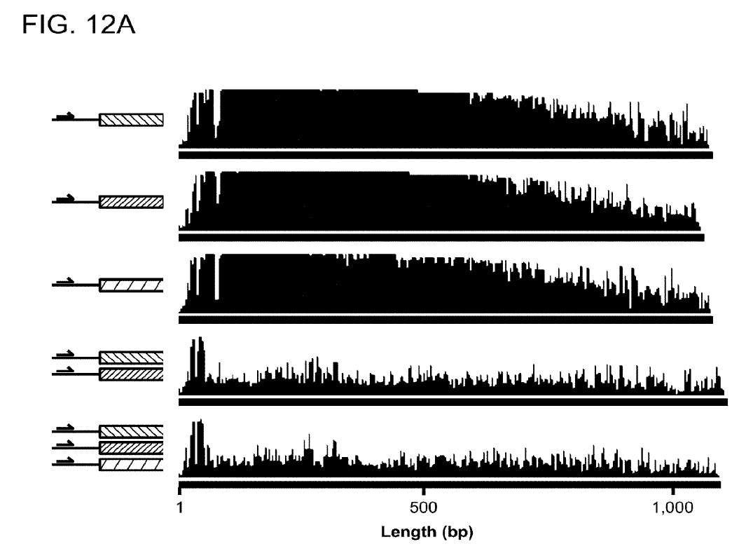

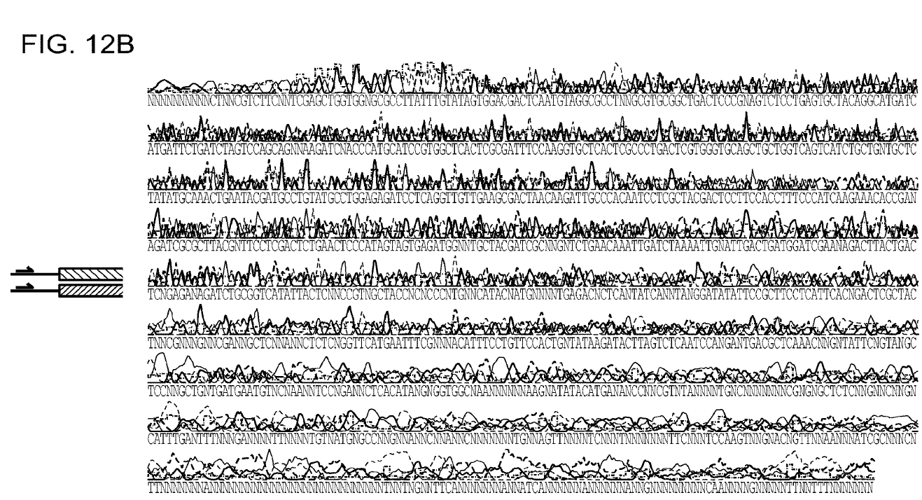

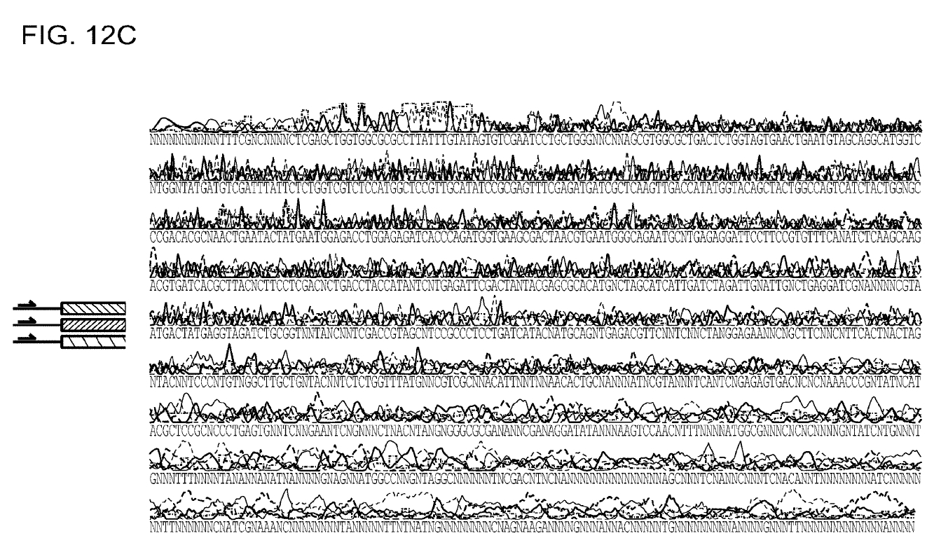

Conventionally, to extract information embedded on multiple DNA strands, one would first have to sequence each strand separately and then perform sequence alignments. In designing MuSE, it was expected that when multiple DNA strands are analyzed together by Sanger sequencing using a common primer, at chromatogram positions where two bases are identical a large peak would be observed and where two bases differ a small peak would be observed, thereby producing a pattern (FIG. 2A). However, the simultaneous sequencing of multiple DNA strands with a common primer cannot be used, as it leads to poor chromatograms and non-specific reads (FIGS. 12A-12C). Chromatogram patterning is based on the rational design of iKey-64 (Tables 2-3), where the aim was to reduce the incidence of homopolymers in DNA messages as long stretches of homopolymers lead to sequencing inaccuracies¹⁷. The homopolymer codons AAA, CCC, GGG, and TTT are assigned to four function keys, ensuring that in normal text no homopolymer longer than four bases is possible. Even letter combinations yielding four identical bases (such as GTT-TTC representing V-K on the keyboard) are kept quite rare. Therefore, the codon assignment of iKey-64 was based on the frequency of use of letters in the English language¹⁸ to minimize the occurrence of homopolymers and achieve chromatogram patterning.

As shown in Table 3, the buttons of this embodiment of the iKey-64 were separated in to 3 categories based on the frequency of use as judged by qualitative measures. Category 1 is for the most frequently used buttons and is encoded by codons that contain three different nucleotides. Category 2 is for less frequently used buttons and is encoded by codons that contain the same nucleotide in the first and third position. Category 3 is for the least frequently used buttons and is encoded by codons that contain two or more homopolymers. Since iKey-64 is similar in design to a one-time pad, many possible versions exist and the last column provides the number of potential permutations that exist for randomly shuffling the codons between the buttons. The frequency of letters in the English alphabet were based on Table 2. If chromatogram patterning is not desired, then all 64 buttons in iKey-64 can be randomly shuffled for transcription of plaintext on to DNA.

TABLE 2
Rational Design of iKey-64:
Letter Frequency

| Letter | Frequency |
|---|---|
| E | 11.1607% |
| A | 8.4966% |
| R | 7.5809% |
| I | 7.5448% |
| O | 7.1635% |
| T | 6.9509% |
| N | 6.6544% |
| S | 5.7351% |
| L | 5.4893% |
| C | 4.5388% |
| U | 3.6308% |
| D | 3.3844% |
| P | 3.1671% |
| M | 3.0129% |
| H | 3.0034% |
| G | 2.4705% |
| B | 2.0720% |
| F | 1.8121% |
| Y | 1.7779% |
| W | 1.2899% |
| K | 1.1016% |
| V | 1.0074% |
| X | 0.2902% |
| Z | 0.2722% |
| J | 0.1965% |
| Q | 0.1962% |

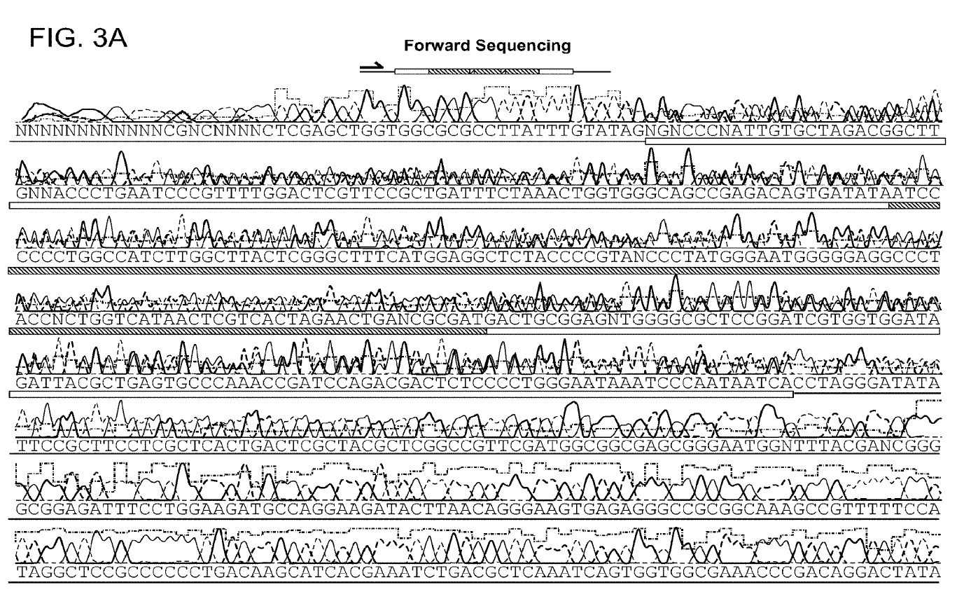

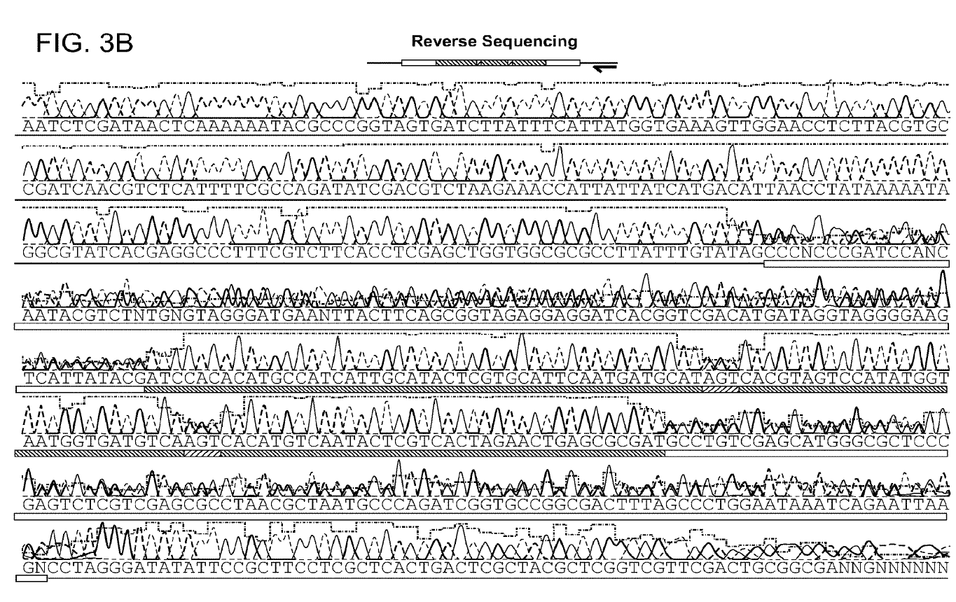

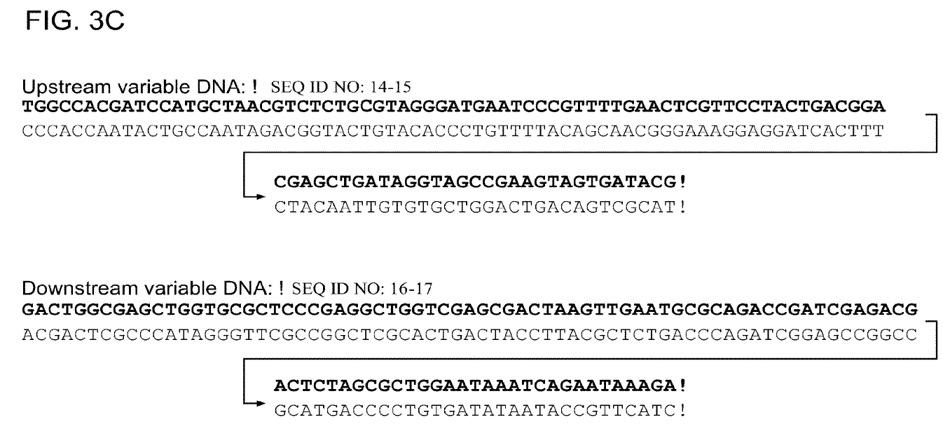

iKey-64 was tested for MuSE by writing the cipher ‘Massachusetts Institute Technology’ on two DNA strands, where “space1” (AGT) was used in DNA-1 and “space2” (CTA) with DNA-2 to demarcate individual words in the sequences (FIGS. 2B-2C). Co-sequencing both DNA samples together would introduce troughs around words in the chromatogram. Individual sequencing of DNA-1 and DNA-2 produced high quality reads, however in a DNA-1+2 mixture forward sequencing with a common primer did not produce chromatogram patterning, but rather camouflaged the cipher (FIG. 2D). This was due to the variable DNA sequences placed upstream of the ciphers, where stretches of C and A homopolymers at the 5′ ends interfered with base determination during Sanger sequencing causing intentional misalignment of the recognized bases in the chromatogram (FIGS. 3A-3C). On the other hand, reverse sequencing of DNA-1+2 with a common primer produced a distinct pattern on the chromatogram. Since there were no interfering stretches of homopolymers in the variable DNA regions, there were no shifts in the base identities during sequencing leading to predictable chromatogram patterning and a single-step extraction of information from the two strands (FIGS. 3B-3C).

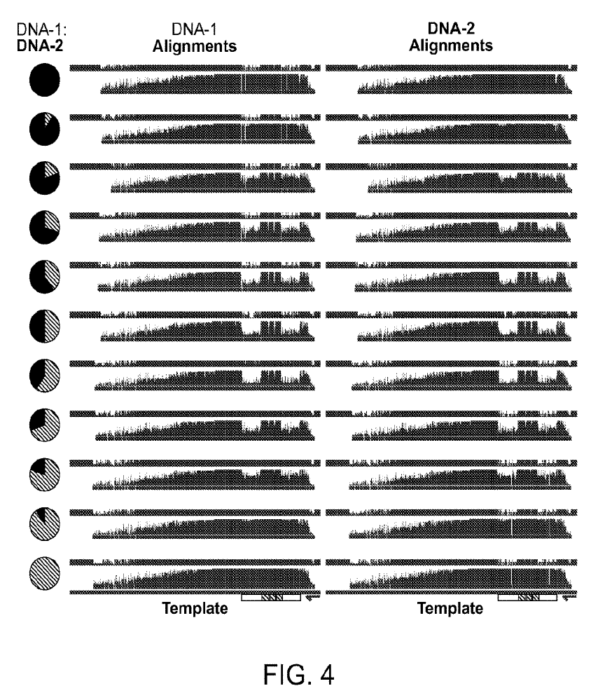

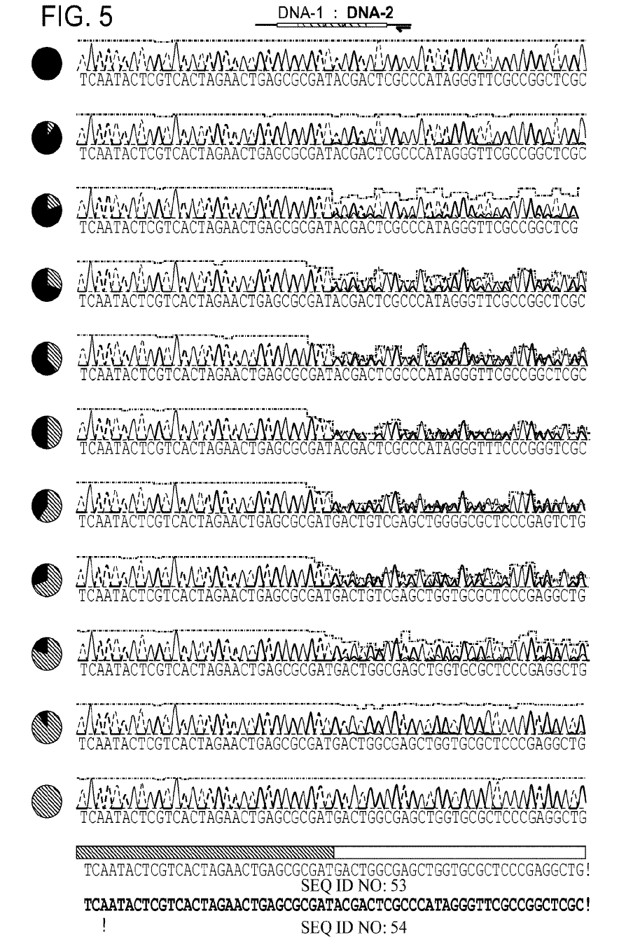

MuSE can be tuned to embed data in chromatograms discreetly so that sequence alignments derived from chromatograms cannot be used to identify embedded information. Adjusting the ratio of DNA-1/DNA-2 allows the degree of contrast achieved in the chromatogram patterns to be varied (FIG. 2E). When DNA-1 or DNA-2 is present at 10-30%, chromatogram patterning is still achieved upon close examination of individual peaks, but the resulting sequence produced is only that of the more concentrated partner (FIGS. 4-5). Therefore, an unauthorized user would be unable to see embedded messages directly in the sequence output or in alignments.

### Multiplexed Sequencing of Strand Combinations

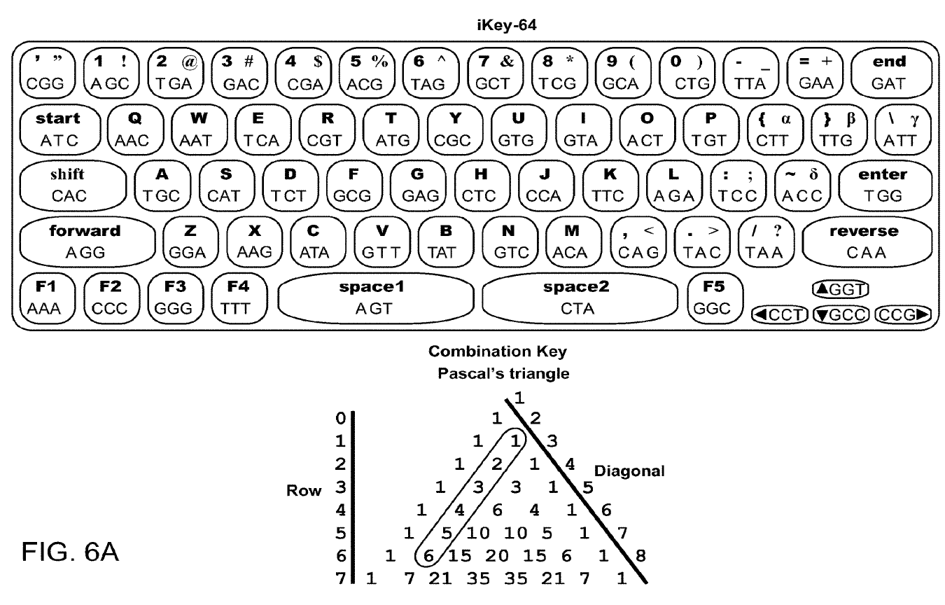

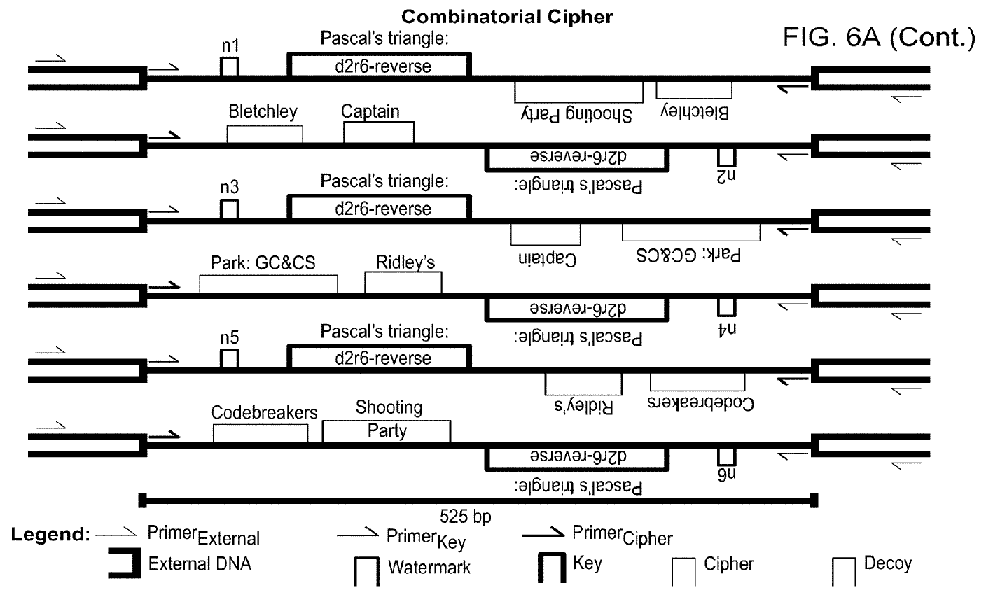

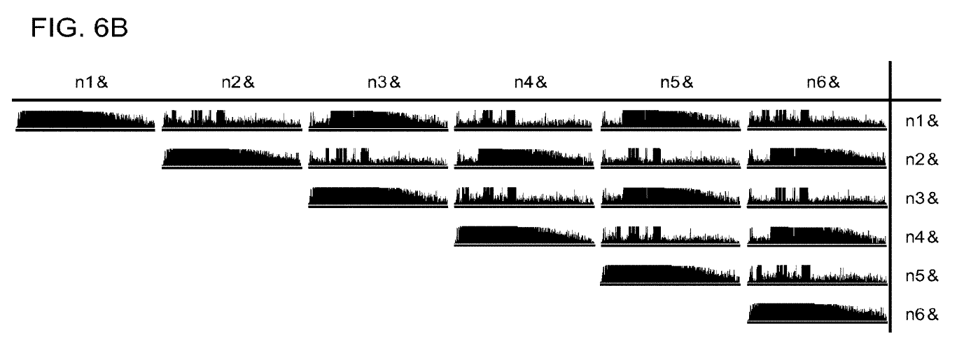

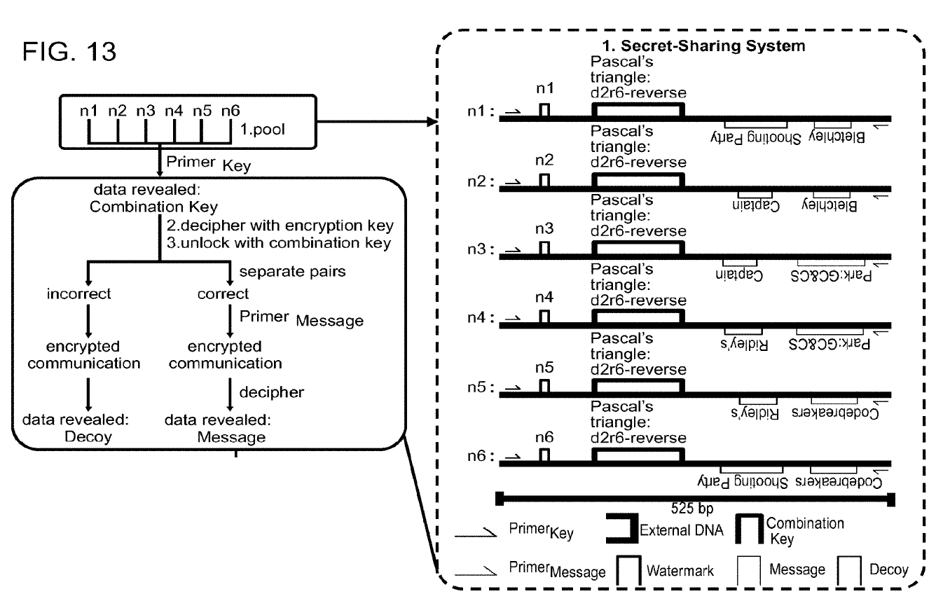

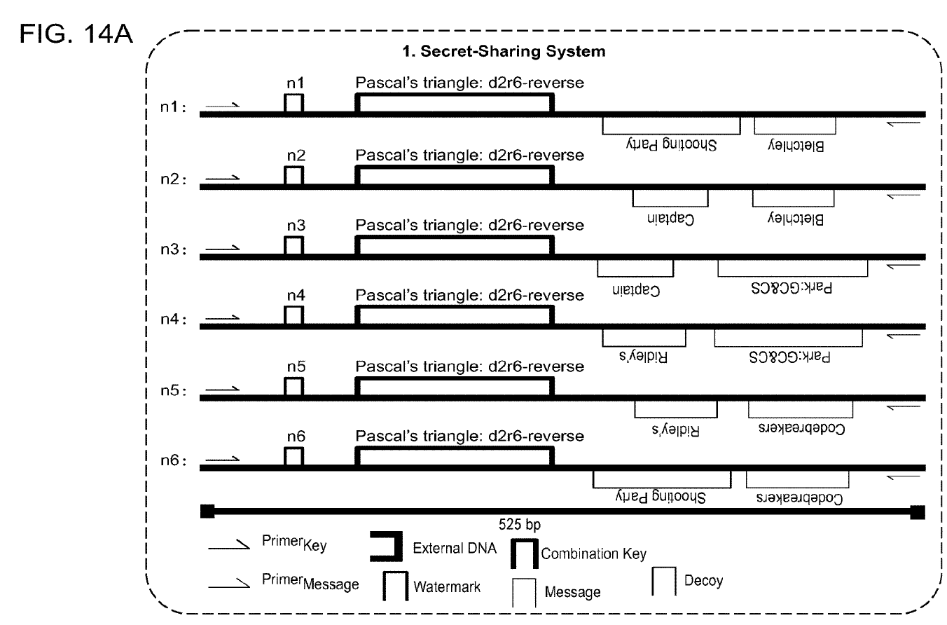

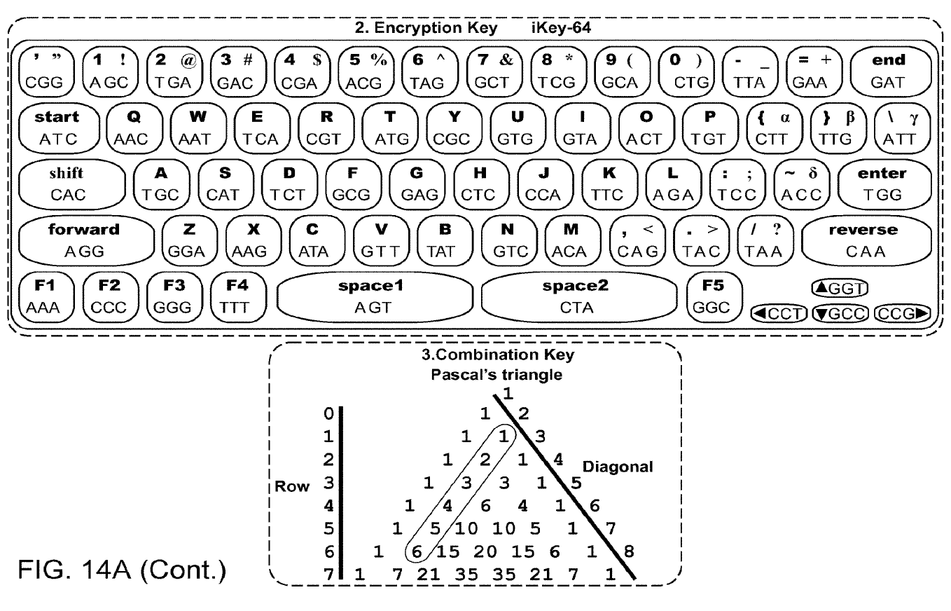

For additional security, MuSE can be used to disseminate information across many DNA strands, where multiplexed sequencing of different strand combinations will provide different readouts (FIG. 13). To demonstrate this, watermarks, a key, a cipher, and a decoy message were encoded across six strands in a 525 bp region of DNA to recreate a World War II communication made during the establishment of Bletchley Park (FIG. 6A and FIG. 14)¹⁹. The functions of the elements are: (i) watermarks—an identification tag for each strand, (ii) key—a riddle whose solution would provide the correct strand combinations required for co-sequencing to reveal the cipher in the secret-sharing system, (iii) cipher—the desired message to be communicated, and (iv) decoy—a false message to be revealed if improper strand combinations were used for co-sequencing.

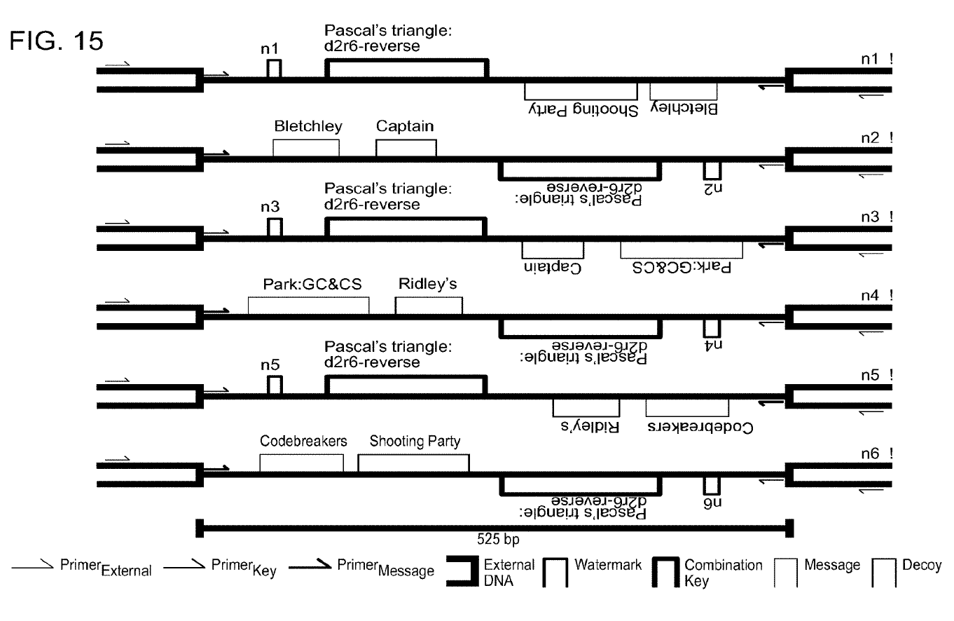

To extract the information via co-sequencing, two different primers—Primer_{Key} and Primer_{Cipher}—that are common to all six strands are required. As a demonstration for this exercise a simple key was chosen, where co-sequencing of all of the strands with Primer_{Key} revealed the message: Pascal's triangle: d2r6-reverse (FIG. 6A). This serves as a combination key and means the cipher is revealed from pairs as ordered is Pascal's triangle diagonal 2 down until row 6 on the reverse strand. If strand pairs n1+2, n3+4, and n5+6 were to be co-sequenced using Primer_{Cipher}, then the embedded message ‘Bletchley Park: GC&CS Codebreakers’ would be revealed. However, if one were to for example misinterpret the key, then a decoy message could be revealed. Here, one decoy message was embedded—‘Captain Ridley's Shooting Party’—hat would be revealed if one were to co-sequence pairs n2+3, n4+5, and n6+1, a circular permutation of the key. Of course, more than one decoy message could be embedded to further introduce complexity in communications. Alternatively, an unauthorized user may use random primers—Primer_{ExternalFw/Rv}—instead of Primer_{Key} and Primer_{Cipher} to extract messages if they were embedded in large DNA regions. To obfuscate this approach, the embedded information was alternated between the forward and reverse strands to provide a camouflage effect (FIG. 15. Since any secure communication would have a limited quantity of DNA (enough to extract the desired message once), an unauthorized user would be unable to exhaustively explore primer sequences to extract information without advanced scientific protocols.

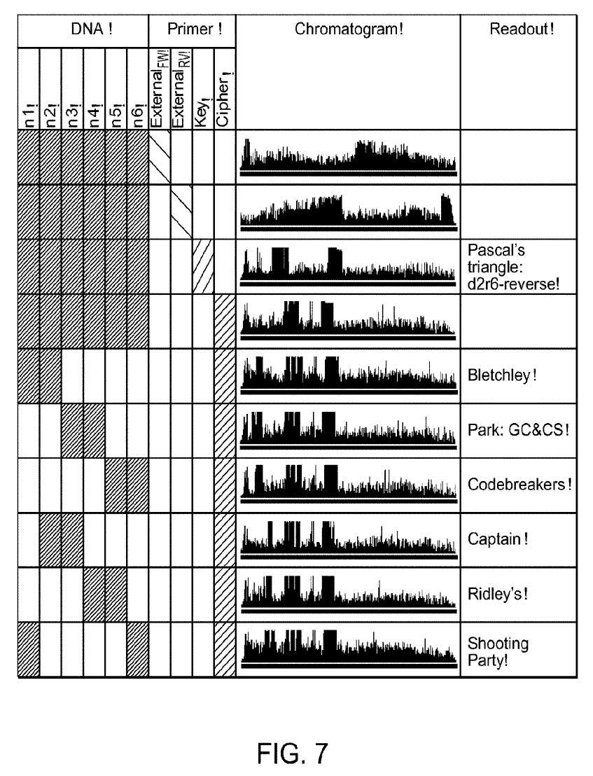

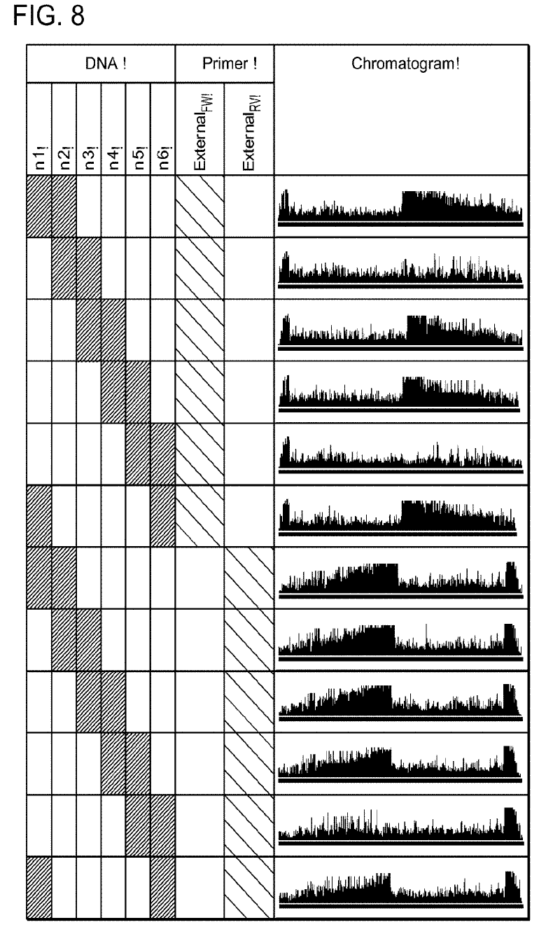

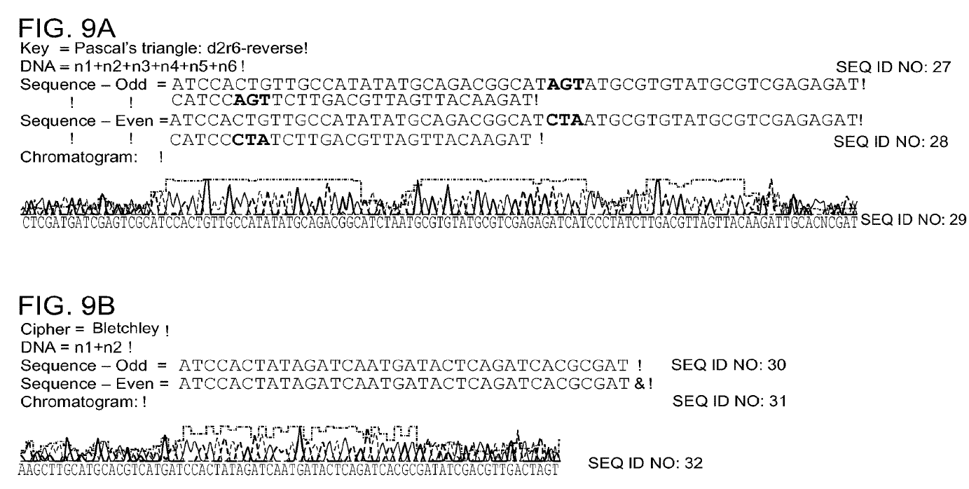

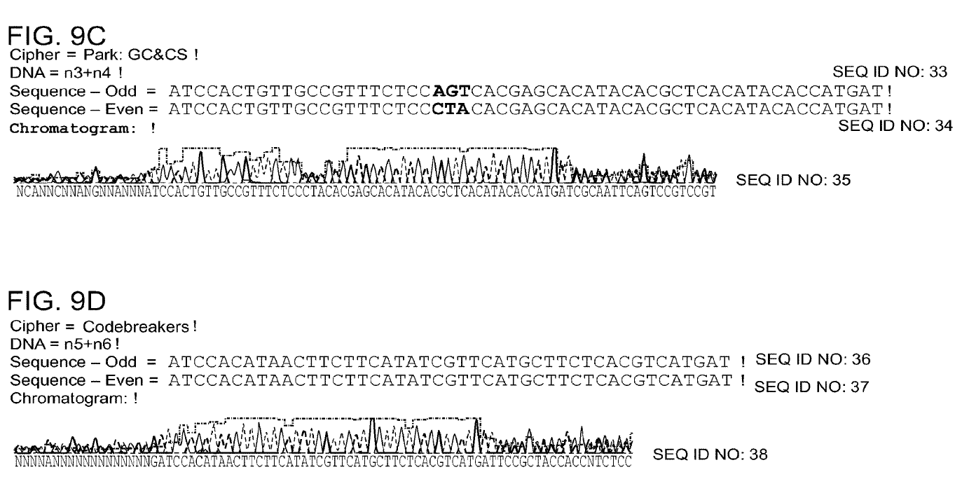

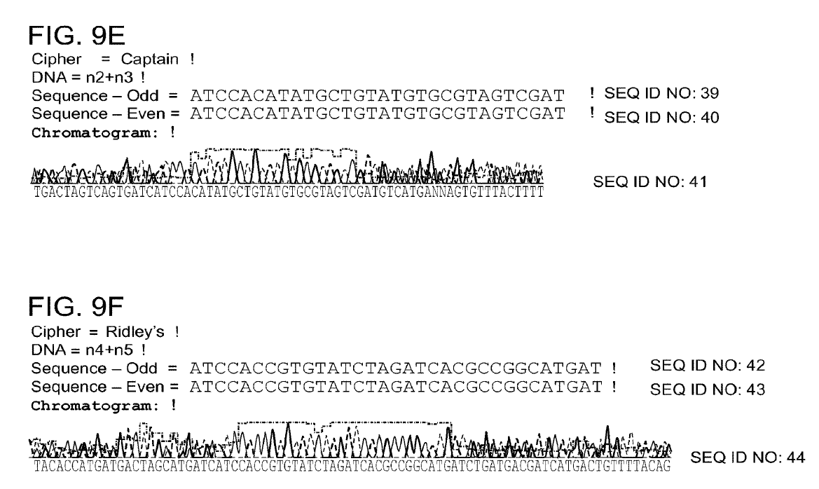

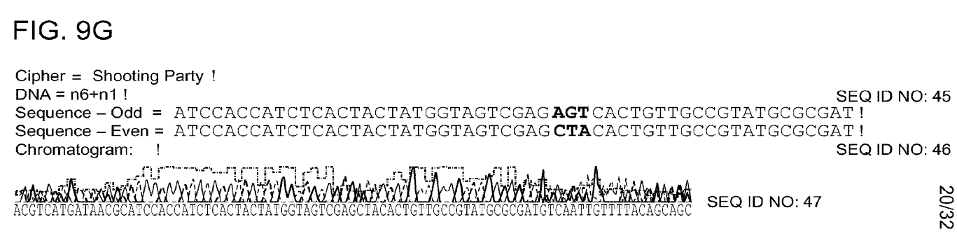

As expected, co-sequencing with Primer_{ExternalFw/Rv} did not produce chromatogram patterning, whether cipher/decoy pairs or all six strands were co-sequenced (FIGS. 7-8). However, co-sequencing of all six strands with Primer_{Key} produced the readout ‘Pascal's triangle: d2r6-reverse’, while the cipher/decoy containing regions did not produce chromatogram patterning. Similarly, chromatogram patterning was not observed in the cipher/decoy containing regions when Primer_{Cipher} was used for co-sequencing all six strands. On the other hand, sequencing of pairs with Primer_{Cipher} as per the order in Pascal's triangle—n1+2, n3+4, and n5+6—revealed the cipher via chromatogram patterning (FIGS. 9A-9G). Similarly, co-sequencing of the incorrect pairs—n2+3, n4+5, and n6+1—led to a decoy message to be revealed. Expectedly, co-sequencing of other pair combinations did not lead to any patterning (FIG. 6B). This demonstrated that in addition to the security afforded by iKey-64 and MuSE, one must also decipher the key accurately to unlock embedded messages.

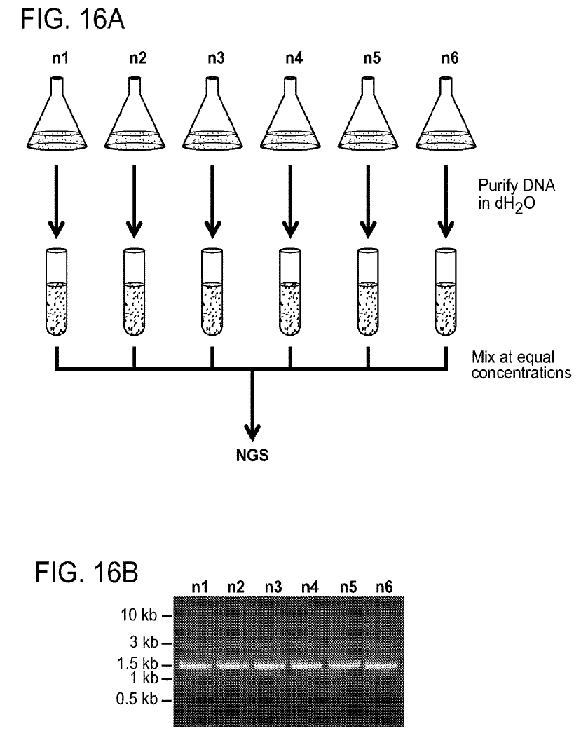

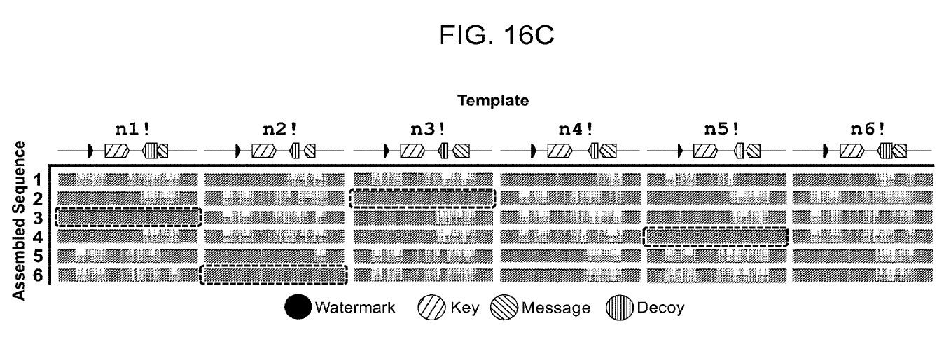

If unauthorized individuals were to gain access to a DNA communication, next-generation sequencing (NGS) might also be attempted for extracting messages. To recreate such a scenario, the difficulty associated with NGS analysis of unknown DNA samples was tested. A purified mixture of DNA samples n1+n2+n3+n4+n5+n6 was prepared and submitted for NGS analysis to an outside party under blind experimental conditions, with a request to provide the assembled contents of the sample (FIG. 16A-16B). While sequencing of the mixture produced ˜2 million reads, the blind assembly of the reads to reconstruct the contents proved difficult and inconclusive (Table 4). However, after the initial analysis the outside party was informed that there were 6 plasmids in the sample, each containing 525 bp messages as inserts. The vector sequence was then provided and the outside party asked for the exact sequences of the messages in the sample. A second round of analysis identified 6 assembled sequences that represented our messages (Table 5). Alignment of the 6 identified sequences with n1, n2, n3, n4, n5, and n6 templates provided most of the information in the six messages, with n1, n2, n3, and n5 providing almost perfect alignments (FIG. 16C). This demonstrated the difficulty associated with blind sequencing of a MuSE communication without any prior knowledge of DNA contents. Even if the sequences of a DNA communication were identified after considerable time and expense, the contents of a communication would still likely be protected by the iKey, combination key, and decoy/non-coding sequences.

TABLE 4
Next-generation sequencing statistics of assembled reads
under blind experimental conditions.

| | n1 + n2 + n3 + n4 + n5 + n6 |
|---|---|
| Sequence size | 1,407,947 |
| Number of scaffolds | 2,851 |
| % GC | 51.1 |
| Shortest contig size | 300 |
| Median sequence size | 423 |
| Mean sequence size | 493.8 |
| Longest contig size | 4,625 |
| Number of subsystems | 22 |
| Number of coding sequences | 984 |
| Number of RNAs | 0 |

*NGS sequencing of a mixture of samples n1 + n2 + n3 + n4 + n5 + n6 (FIG. S10) produced 1,997,179 reads at 300 bp with 47% GC content. Shown are the statistics of the assembled scaffolds by the MIT BioMicro Center under blind experimental conditions. While the DNA samples produced high quality reads, under blind experimental conditions assembly of the reads in to the original constructs proved challenging and the results were inconclusive. n1 = 2,346 bp/47.4% GC, n2 = 2,346 bp/47.3% GC, n3 = 2,346 bp/47.5% GC, n4 = 2,346 bp/47.6% GC, n5 = 2,346 bp/47.4% GC, n6 = 2,346 bp/47.3% GC.

TABLE 5
Identified sequences from NGS analysis.

| Assembled | | |
|---|---|---|
| Sequence | Sequence | SEQ ID NO: |
| 1 | TAATACGACTCACTATAGGGACAGTCTAGTGCAGCAGTCAGTACGAGTCT | 18 |
| | CATGAGTGTAGGATGCATGAGATCAACGCTAGCATCGCACTGTCGTCATG | |
| | CAGCTGACTCCGATCTGACTATCGTCTGAGATCAGAGCGTAACGTAGTCA | |
| | GTGCTAGCATGCGAACTCGATGATCGAGTCGTATCCACTGTTGCCATATA | |
| | TGCAGACGGCATAGTATGCGTGTATGCGTCGAGAGATCATCCCTATCTTG | |
| | ACGTTAGTTACAAGATCCCACCAATACTGCCAATAGACGGTCCTCCTTTC | |
| | CCGTTGCTGTAAAACAGTCATGATCGTCATCAGATCATGCCGGCGTGATC | |
| | TAGATACACGGTGGATTCAGCTACTAGTCGAATCATGACGTGAGAAGCAT | |
| | GAACGATATGAAGAAGTTATGTGGATAGCTGTCGACGTGATCGTATCGAT | |
| | GCAGTCCTCAGGTCATATTACTCGACAGTTGCTAAGTCAGTCATCGTCAT | |
| | ACGATGCCGCTGAGCAATAACTAGC | |
| 2 | TAATACGACTCACTATAGGGACAGTCTAGTGCAGCAGTCAGTACGAGTCT | 19 |
| | CATGAGTGTAGGATGCATGATCATGATTCTGATCTAGTCCAGCAGTAGAG | |
| | TCGTCTCGATCGATCTGTGCATCGTCAGCGATATTCGACGTAGTCGCTCG | |
| | ACCTGACTCGTGAGTGCAGCTACGTGTCAGTCATCCACTGTTGCCATATA | |
| | TGCAGACGGCATAGTATGCGTGTATGCGTCGAGAGATCATCCAGTTCTTG | |
| | ACGTTAGTTACAAGATTGGCCACGATCCATGCTAACGTCTCTTCCACCTT | |
| | TCCCAAAAAGTAACACACCATGACGTATCGACTACGCACATACAGCATAT | |
| | GTGGATGATCACTGACTGACTGAACTACGATCATGGTGTATGTGAGCGTG | |
| | TATGTGCTCGTGACTGGAGAAACGGCAACAGTGGATGATTGACGTACGAC | |
| | TGCTAGCTCAGGTCATATTACTCGACAGTTGCTAAGTCAGTCATCGTCAT | |
| | ACGATGCCGCTGAGCAATAACTAGC | |
| 3 | TAATACGACTCACTATAGGGACAGTCTAGTGCAGCAGTCAGTACGAGTCT | 20 |
| | CATGAGTGTAGGATGCATGATCATGATTCTGATCTAGTCCAGCAGTAGAG | |
| | TCGTCTCGATCGATCTGTGCATCGTCAGCGATATTCGACGTAGTCGCTCG | |
| | ACCTGACTCGTGAGTGCAGCTACGTGTCAGTCATCCACTGTTGCCATATA | |
| | TGCAGACGGCATAGTATGCGTGTATGCGTCGAGAGATCATCCAGTTCTTG | |
| | ACGTTAGTTACAAGATTGGCCACGATCCATGCTAACGTCTCTTCCACCTT | |
| | TCCCAAAAAGTAACACCGACTGATCGCGCATACGGCAACAGTGACTCTCG | |
| | ACTACCATAGTAGTGAGATGGTGGATTACGATCGCGTGATCTGAGTATCA | |
| | TTGATCTATAGTGGATTGACTGATGATCGTACTGTCGTACTGACTCTGAC | |
| | GTCGATCTCAGGTCATATTACTCGACAGTTGCTAAGTCAGTCATCGTCAT | |
| | ACGATGCCGCTGAGCAATAACTAGC | |
| 4 | TAATACGACTCACTATAGGGACAGTCTAGTGCAGCAGTCAGTACGAGTCT | 21 |
| | CATGAGTGTAGGATGCATGATCATGATTCTGATCTAGTCCAGCAGTAGAG | |
| | TCGTCTCGATCGATCTGTGCATCGTCAGCGATATTCGACGTAGTCGCTCG | |
| | ACCTGACTCGTGAGTGCAGCTACGTGTCAGTCATCCACTGTTGCCATATA | |
| | TGCAGACGGCATAGTATGCGTGTATGCGTCGAGAGATCATCCAGTTCTTG | |
| | ACGTTAGTTACAAGATTGGCCACGATCCATGCTAACGTCTCTTCCACCTT | |
| | TCCCAAAAAGTAACACTGACTGCATTCGTGATCATCATGCCGGCGTGATC | |
| | TAGATACACGGTGGATTCAGCTACTAGTCGAATCATGACGTGAGAAGCAT | |
| | GAACGATATGAAGAAGTTATGTGGATAGCTGTCGACGTGATCGTATCGAT | |
| | GCAGTCCTCAGGTCATATTACTCGACAGTTGCTAAGTCAGTCATCGTCAT | |
| | ACGATGCCGCTGAGCAATAACTAGC | |
| 5 | TAATACGACTCACTATAGGGACAGTCTAGTGCAGCAGTCAGTACGAGTCT | 22 |
| | CATGAGTGTAGGATGCATGAGATCAACGCTAGCATCGCACTGTCGTCATG | |
| | CAGCTGACTCCGATCTGACTATCGTCTGAGATCAGAGCGTAACGTAGTCA | |
| | GTGCTAGCATGCGAACTCGATGATCGAGTCGTATCCACTGTTGCCATATA | |
| | TGCAGACGGCATAGTATGCGTGTATGCGTCGAGAGATCATCCCTATCTTG | |
| | ACGTTAGTTACAAGATCCCACCAATACTGCCAATAGACGGTCCTCCTTTC | |
| | CCGTTGCTGTAAAACATAGTCATGACATCGACTACGCACATACAGCATAT | |
| | GTGGATCTAGCTTGACTAGTCAACGTCGATATCGCGTGATCTGAGTATCA | |
| | TTGATCTATAGTGGATTGACTGATGATCGTACTGTCGTACTGACTCTGAC | |
| | GTCGATCTCAGGTCATATTACTCGACAGTTGCTAAGTCAGTCATCGTCAT | |
| | ACGATGCCGCTGAGCAATAACTAGC | |
| 6 | TAATACGACTCACTATAGGGACAGTCTAGTGCAGCAGTCAGTACGAGTCT | 23 |
| | CATGAGTGTAGGATGCATGAGATCAACGCTAGCATCGCACTGTCGTCATG | |
| | CAGCTGACTCCGATCTGACTATCGTCTGAGATCAGAGCGTAACGTAGTCA | |
| | GTGCTAGCATGCGAACTCGATGATCGAGTCGTATCCACTGTTGCCATATA | |
| | TGCAGACGGCATAGTATGCGTGTATGCGTCGAGAGATCATCCCTATCTTG | |
| | ACGTTAGTTACAAGATCCCACCAATACTGCCAATAGACGGTCCTCCTTTC | |
| | CCGTTGCTGTAAAACATAGTCATGACATCGACTACGCACATACAGCATAT | |
| | GTGGATCTAGCTTGACTAGTCAACGTCGATATCGCGTGATCTGAGTATCA | |
| | TTGATCTATAGTGGATCATGACGTGCATGCAAGCTTAGCTAGTCAGATCA | |
| | GTAGCTCTCAGGTCATATTACTCGACAGTTGCTAAGTCAGTCATCGTCAT | |
| | ACGATGCCGCTGAGCAATAACTAGC | |

*After blind analysis by the MIT BioMicro Center did not provide the contents of
the unknown sample submitted for analysis, further information about the plasmids
and vector sequences were provided. Shown here are the 6 assembled and identified
sequences each 525 bp, representing the messages encoded in n1, n2, n3, n4, n5,
and n6 generated by the MIT BioMicro Center after a second round of analysis.
Alignments to n1, n2, n3, n4, n5, and n6 are in FIG. 16C.

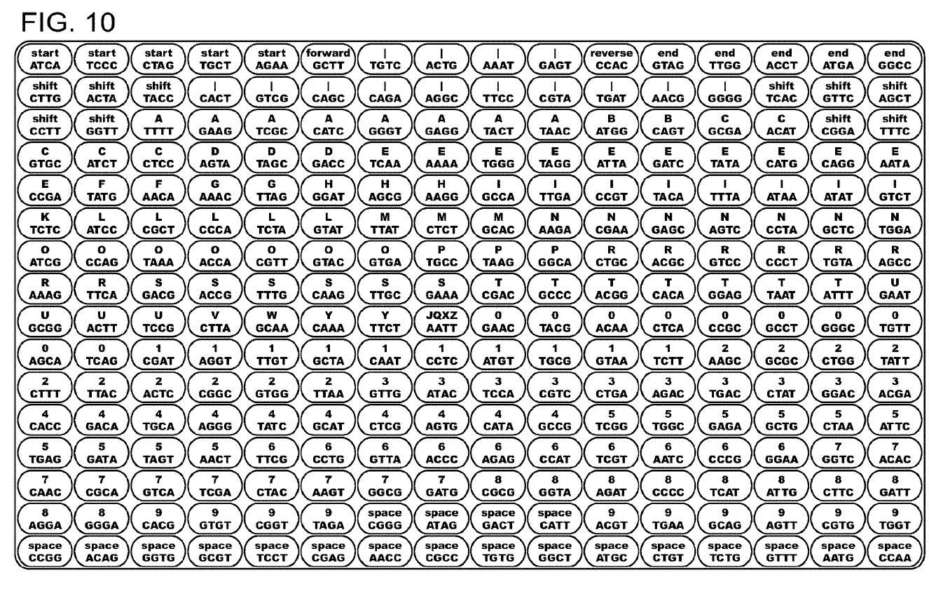

iKey-64 is designed to convert plaintext in to a DNA encodable language. If chromatogram patterning is desired, the codons may potentially be shuffled to enable 9.1×10⁶¹ variants (Table 3). However, if chromatogram patterning is not desired then a maximum of 1.3×10⁸⁹ variants exist, significantly increasing the security of encoded information. As a communication medium, knowledge of the appropriate primers, combination key, and incorporation of decoy messages would also provide additional data security. Nevertheless, data encoded using iKey-64 would still not be truly random due to the frequency of use for each button, but additional measures may be implemented to increase security: (i) Cryptography plaintext information may first be subject to advanced cryptographic algorithms, (ii) Linguistics—principles of linguistics may be applied to the layout of iKeys to modify alphabets for DNA communication, introduce new grammar rules or create iKeys in different languages, and (iii) Codons—increasing the number of nucleotides per codon can introduce redundancies in the buttons to adjust for character usage frequency. To illustrate, four nucleotides codons can be used to create a 256 button keyboards such as iKey-256 (FIG. 10). When the number of buttons for each letter is adjusted to reflect its frequency in English text, then the probability of using a button for E would equal Q. Similar redundancies may also be introduced for buttons representing numerals, grammar, and other user-defined functions. For instance, the frequency of numerals may be adjusted according to Benford's Law²⁰.

To further extend the iKey system, codons can be used to represent words or phrases in addition to characters. It is estimated that the vocabulary of an educated native English speaking adult consists of ˜17,000 lemmas, while only 10 lemmas constitute 25% of the words used in English^{21, 22}. Using 8-nucleotide codons could generate iKeys with 65,536 buttons, sufficient to include all of the commonly used words in English as well as accommodate individual letters, numerals, grammatical characters, functional characters, and high frequency words. Theoretically, the iKey platform may be designed to incorporate the entire English language. The Oxford English Dictionary (OED), the most comprehensive record of the English language, contains 291,500 entries and a total of 615,100 word forms²³. To encode all of the entries of the OED on an iKey would require 10-nucleotide codons to generate a 1,048,576 button keyboard. Additionally, the dictionary is composed of 59 million words containing 350 million characters resulting in 5.9 characters/word. This would require 18 nucleotides to encode with an iKey-64 but only 10 nucleotides for an iKey-1,048,576, representing a 44% reduction in DNA requirements.

### REFERENCES

1. Bancroft, C., Bowler, T., Bloom, B. & Clelland, C. T. Long-term storage of information in DNA. Science 293, 1763-1765 (2001).
2. Clelland, C. T., Risca, V. & Bancroft, C. Hiding messages in DNA microdots. Nature 399, 533-534 (1999).
3. Church, G. M., Gao, Y. & Kosuri, S. Next-generation digital information storage in DNA. Science 337, 1628 (2012).
4. Liss, M. et al. Embedding permanent watermarks in synthetic genes. PLoS One 7, e42465 (2012).
5. Cox, J. P. Long-term data storage in DNA. Trends Biotechnol. 19, 247-250 (2001).
6. Sennels, L. & Bentin, T. To DNA, all information is equal. Artif. DNA PNA XNA 3, 109-111 (2012).
7. Haughton, D. & Balado, F. BioCode: two biologically compatible Algorithms for embedding data in non-coding and coding regions of DNA. BMC Bioinformatics 14, 121-2105-14-121 (2013).
8. Heider, D. & Barnekow, A. DNA-based watermarks using the DNA-Crypt algorithm. BMC Bioinformatics 8, 176 (2007).
9. Tulpan, D., Regoui, C., Durand, G., Belliveau, L. & Leger, S. HyDEn: a hybrid steganocryptographic approach for data encryption using randomized error-correcting DNA codes. Biomed. Res. Int. 2013, 634832 (2013).
10. Kawano, T. Run-length encoding graphic rules, biochemically editable designs and steganographical numeric data embedment for DNA-based cryptographical coding system. Commun. Integr. Biol. 6, e23478 (2013).
11. Ekert, A. & Renner, R. The ultimate physical limits of privacy. Nature 507, 443-447 (2014).
12. Gehani, A., LaBean, T. & Reif, J. DNA-based Cryptography. DNA Based Computers V: Dimacs Workshop DNA Based Computers V Jun. 14-15, 1999 Massachusetts Institute of Technology 54, 233 (2000).
13. Mao, C., LaBean, T. H., Relf, J. H. & Seeman, N. C. Logical computation using algorithmic self-assembly of DNA triple-crossover molecules. Nature 407, 493-496 (2000).
14. Hirabayashi, M., Kojima, H. & Oiwa, K. in (eds Peper, F., Umeo, H., Matsui, N. & Isokawa, T.) 174-183 (Springer Japan, 2010).
15. Hirabayashi, M., Kojima, H. & Oiwa, K. Effective algorithm to encrypt information based on self-assembly of DNA tiles. Nucleic Acids Symp. Ser. (Oxf) (53):79-80. doi, 79-80 (2009).
16. Voelkerding, K. V., Dames, S. A. & Durtschi, J. D. Next-generation sequencing: from basic research to diagnostics. Clin. Chem. 55, 641-658 (2009).
17. http://www.oxforddictionaries.com/us/words/what-is-the-frequency-of-the-letters-of-the-alphabet-in-english.
18. Ferguson, N., Schneier, B. & Kohno, T. in Cryptography engineering: design principles and practical applications (Wiley Publishing, Inc., Indianapolis, 2010).
19. http://www.bletchleypark.org.uk/.
20. Alves, A. D., Yanasse, H. H. & Soma, N. Y. Benford's Law and articles of scientific journals: comparison of JCR and Scopus data. Scientometrics 98, 173-184 (2014).
21. http://www.oxforddictionaries.com/us/words/the-oec-facts-about-the-language.
22. Goulden, R., Nation, I. S. P. & Read, J. How large can a receptive vocabulary be? Applied Linguistics 11, 341-363 (1990).
23. http://public.oed.com/history-of-the-oed/dictionary-facts/.
24. Gibson, D. G. Enzymatic assembly of overlapping DNA fragments. Methods Enzymol. 498, 349-361 (2011).

What is claimed is:

1. A method of secure communication of information contained on a single nucleic acid molecule, the method comprising:
(a) obtaining a nucleic acid molecule of known sequence;
(b) obtaining a modified keyboard comprising a personalized platform for translating nucleic acid sequence into text; and,
(b) generating a quantum of information translated from the nucleic acid sequence using the modified keyboard of (a).

2. A method of secure communication of information disseminated across at least one nucleic acid molecule, the method comprising:
(a) obtaining a modified keyboard comprising a personalized platform for translating text into a nucleic acid sequence;
(b) translating a quantum of information into a nucleic acid message sequence using the modified keyboard of (a); and,
(c) obtaining a at least one nucleic acid molecules, each molecule comprising (i) the complete or a portion of the nucleic acid message sequence and (ii) at least one contiguous stretch of randomized variable nucleic acid sequence flanking and/or inserted into the message sequence, thereby producing a nucleic acid molecule or a set of nucleic acid molecules containing the entire quantum of information.

3. The method of claim 1 or claim 2, wherein the modified keyboard comprises codons.

4. The method of claim 3, wherein the codons are designed to normalize frequency of character usage.

5. The method of any one of claims 1 to 4, further comprising sequencing the nucleic acid molecule or set of nucleic acid molecules using one or more common primers.

6. The method of claim 5, wherein the sequencing produces a chromatogram.

7. The method of claim 5, wherein the sequencing produces data that is analyzed by sequence alignment or bioinformatics methods.

8. The method of claim 6, further comprising identifying nucleic acid sequence corresponding to areas of high intensity peaks on the chromatogram.

9. The method of claim 6, further comprising identifying nucleic acid sequence corresponding to areas of low intensity peaks on the chromatogram.

10. The method of any one of claims 6-9, further comprising extracting the quantum of information contained within the set of nucleic acid molecules by using the modified keyboard to translate the nucleic acid sequence identified in any one of claims 6-9.

11. The method of any one of claims 1-10, wherein the modified keyboard comprises homopolymer codons located on functional keys.

12. The method of any one of claims 1-11, wherein the codons are greater than 3 nucleotides in length.

13. The method of claim 12, wherein the codons are 4, or 5, or 6, or 7, or 8, or 9, or 10, or 11, or 12, or 13, or 14, or 15, or 16, or 17, or 18 nucleotide bases in length.

14. The method of any one of claims 1-13, wherein the codons are of mixed lengths.

15. The method of any one of claims 1-14, wherein the variable nucleic acid sequence comprises contiguous homopolymer codons.

16. The method of any one of claims 6-15, wherein the sequencing is performed by Sanger sequencing, bridge PCR, nanopore sequencing, or Next Generation Sequencing.

17. The method of any one of claims 1-16, wherein the at least one nucleic acid molecule is sequenced with at least one common primer.

18. The method of any one of claims 1-17, wherein the nucleic acid molecule(s) are in silico.

19. A method of producing an individualized keyboard for the conversion of plaintext into nucleic acid encodable language, the method comprising:
(a) producing a library of codons;
(b) assigning each member of the library to a different symbol; and
(c) arranging the symbols into an array, thereby producing an individualized keyboard.

20. The method of claim 19, wherein the codons are greater than three nucleotide bases in length.

21. The method of claim 19 or claim 20, wherein the codons are 4, or 5, or 6, or 7, or 8, or 9, or 10, or 11, or 12, or 13, or 14, or 15, or 16, or 17, or 18 nucleotide bases in length.

22. The method of any one of claims 19-21, wherein the codons are of mixed lengths.

23. The method of any one of claims 19-22, wherein the symbol is selected from the group consisting of letter, number, word, punctuation mark, pictogram or logogram.

24. The method of any one of claims 2-18, wherein the variable sequence comprises at least one contiguous stretch of homopolymer codons.

25. The method of any one of claims 19-23, wherein the individualized keyboard comprises homopolymer codons associated only with functional keys.

26. The method of any one of claims 19-23, wherein the codons are designed to normalize frequency of character usage.
